(19) United States
(12) Patent Application Publication
(10) Pub. No.: US 2009/0198088 A1
(43) Pub. Date: Aug. 6, 2009

(54) PROCESS FOR THE PURIFICATION OF CRUDE GLYCERIN UTILIZING ION EXCLUSION CHROMATORGRAPHY AND GLYCERIN CONCENTRATION

(75) Inventors: Anthony P. Tirio, Pittsburgh, PA (US); George R. Gallaher, Oakmont, PA (US); Hans-Karl Soest, Cologne (DE); Rudolf Wagner, Cologne (DE)

Correspondence Address: LANXESS CORPORATION, 111 RIDC PARK WEST DRIVE, PITTSBURGH, PA, 15275-1112, US

(73) Assignee: LANXESS Sybron Chemicals Inc., Birmingham, NJ (US)

(21) Appl. No.: 12/360,585

(22) Filed: Jan. 27, 2009

Related U.S. Application Data
(60) Provisional application No. 61/063,235, filed on Feb. 1, 2008.

(51) Int. Cl. C07C 31/18

(52) U.S. Cl. 568/870

(57) ABSTRACT

A process for the purification of crude glycerin utilizing ion exclusion chromatography fractionation, and one or more dewatering steps under moderate temperatures and pressures.

This application claims priority to provisional U.S. Application No. 61/063,235, Filed Feb. 1, 2008, entitled A PROCESS FOR THE PURIFICATION OF CRUDE GLYCERIN UTILIZING ION EXCLUSION CHROMATOGRAPHY AND GLYCERIN CONCENTRATION, incorporated herein by reference.

### FIELD OF THE INVENTION

The present invention relates generally to a process for purifying crude glycerin, such as that formed as a by-product of biofuels production, as well as to the product of such a process. The process broadly includes the purification of crude glycerin by ion exclusion chromatography, fractionation, and one or more dewatering steps utilizing moderate temperatures and pressures.

### BACKGROUND OF THE INVENTION

In the search for secure sources of transportation fuels, much effort has been directed towards bioethanol and biodiesel. These agriculturally based fuels lessen the dependence on petroleum from foreign sources and have been embraced worldwide. In the production of these biofuels, organic by-products such as crude glycerin are produced. This by-product must be either disposed of or used for other purposes. The magnitude of the volume of crude glycerin produced as a result of biodiesel production makes disposal undesirable and ultimately uneconomical. Crude glycerin from agricultural sources will contain pure glycerol, the valued component in the solution/mixture, water, low boiling organic compounds, non-volatile salts and low volatility organic compounds. There are several well documented uses of refined glycerin as a replacement for petrochemicals but in general, the glycerin quality must be upgraded to remove contaminants.

The traditional method of upgrading crude glycerin involves evaporating glycerol from non-volatile inorganic salts in one or multiple stages then further evaporating the de-ashed glycerin solution from other higher boiling organics. (The terminology for these other organics is MONG—matter, organic, non-glycerin.) The traditional process to purify crude glycerin starts with the evaporation of lower boiling contaminants such as methanol and water. This is a relatively simple unit operation that involves heating the crude glycerin above the atmospheric boiling points of methanol and water (100° C. and 65° C., respectively) and reducing pressure. Moderate heat to supply heat of vaporization is used.

After low boiling volatile contaminants are removed, the remaining glycerin solution can be evaporated so as to reduce non-volatile inorganic salts in a wiped film evaporator. At atmospheric pressure, pure glycerol boils at 290° C. To initiate boiling, a heat source of at least 300° C., such as very high pressure steam or recirculation hot oil, is required. To reduce the high temperature required to boil the glycerin solution, vacuum is applied to lower the boiling point. At 40 mmHg absolute pressure, the boiling point of pure glycerol is 198° C. and high pressure steam, or hot oil, is still required. Because of the solution colligative property of boiling point elevation caused by the salt contaminants in the glycerin solution, the boiling point of glycerol will be higher than the temperatures stated above. To maintain reasonable heat transfer and to remove accumulated salt solids, a wiped film evaporator is required. The wet salt solids are mechanically wiped from the heat transfer surface and directed out a rotating lock valve at the base of the evaporator. The rotating compartment valve is required because of the physical condition of the salt solids. The wiped film evaporator is a complex heat transfer device that is relatively expensive to purchase and install. Additionally, a wiped film evaporator can be expensive to maintain due to complex system of moving parts and mechanical seals.

Following salt removal, the glycerin solution is evaporated from contaminants to obtain desired purity. Again, high temperatures and deep vacuum are required. This heat transfer operation may be carried out in a long tube, thin-film evaporator since the purged contaminants are liquid.

Following thin film evaporation, the glycerin solution product may require additional purification to remove color body contaminants. This decolorization can be accomplished with activated carbon or ion exchange resin.

### BRIEF SUMMARY OF THE INVENTION

There is broadly contemplated, in accordance with at least one presently preferred embodiment of the present invention, a process for purifying crude glycerin comprising one or more of the steps of: a) providing crude glycerin, said crude glycerin comprising glycerol, water, and at least one of methanol, free fatty acids, FAME, and salts; b) fractionating the crude glycerin thereby forming at least a first fraction comprising glycerol and water and a second fraction comprising water and at least one of methanol, free fatty acids, FAME, and salts; c) a first dewatering of the first fraction thereby producing an industrial grade glycerin solution product, said industrial grade glycerin solution product comprising glycerol and water where the glycerol weight percent is 60 to 90 wt %; and d) a second dewatering of the industrial grade glycerin solution product thereby producing a purified grade glycerin solution product comprising glycerol and water where the glycerol weight percent is between 95 to 100 wt %.

Further, in another embodiment of the invention, said fractionation step b) comprises ion exclusion chromatography (hereinafter “IEC”) as a means of separating glycerol from the salts and other by-products of the crude glycerin, where said other by-products include at least one of methanol, free fatty acids, and FAME.

In a further embodiment of the invention the IEC is performed with the use of a single column fixed bed process, a moving bed process, and/or simulated moving bed process.

In another embodiment of the present invention there is contemplated that the second dewatering step comprises adding the industrial grade glycerin solution to a glycerin water stripper apparatus having a bottom, a middle, and a top area, in which recirculating nitrogen gas and/or air is introduced into the bottom and wherein water of the industrial grade glycerin solution is removed from the middle and/or top of the apparatus while the purified grade glycerin solution product is collected and removed from the bottom of the glycerin water stripper apparatus.

Further the second dewatering step may alternatively comprise evaporation of an industrial grade glycerin solution via the use of one or more of a multi-effect vacuum evaporation apparatus, a thermal recompression apparatus, and a reboiled distillation apparatus. For a better understanding of the present invention, together with other and further features and advantages thereof, reference is made to the following description, taken in conjunction with the accompanying drawings, and the scope of the invention will be pointed out in the appended claims.

### BRIEF DESCRIPTION OF DRAWINGS

FIG. 1 schematically illustrates the traditional process for purifying crude glycerin of the prior art, including wiped film evaporation and thin film evaporation.

FIG. 2 schematically illustrates a broad overview of a process in accordance with at least one embodiment of the present invention, including ion exclusion chromatography, crude and industrial grade glycerin solution dewatering steps, and waste water desalination/concentration.

FIG. 3 schematically illustrates a process for biodiesel production, including a typical continuous transesterification reaction system with phase separation of crude glycerin.

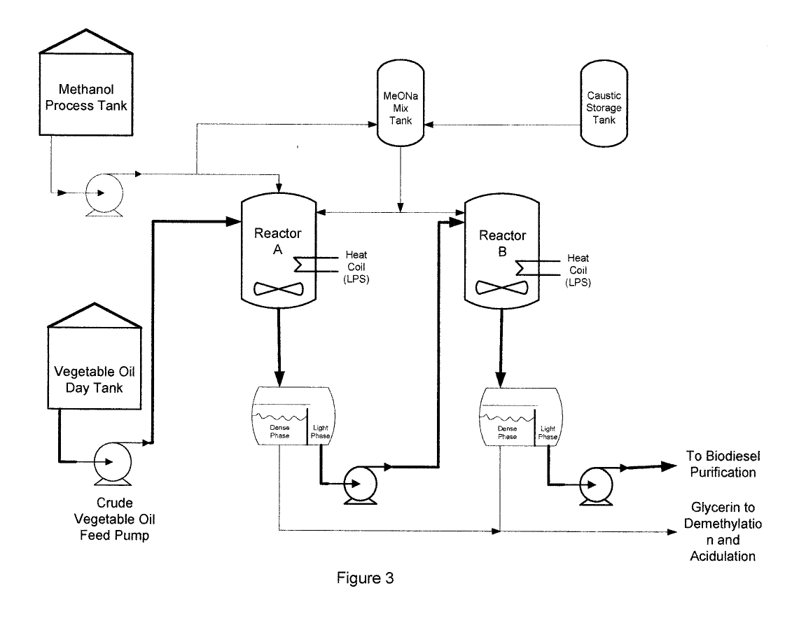

FIG. 4 continues the schematic illustration of the process of biodiesel production as shown in FIG. 3, including biodiesel purification by demethylation and biodiesel purification with ion exchange resin.

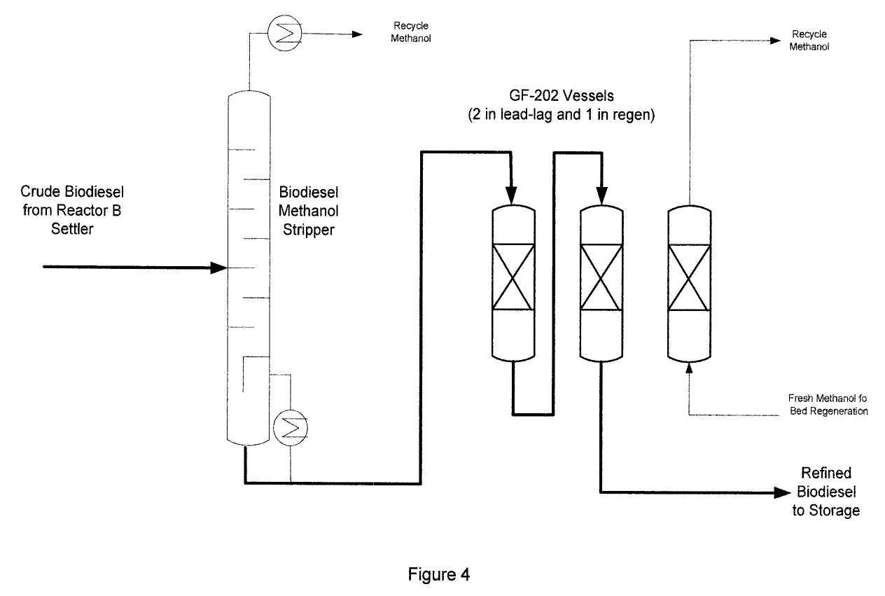

FIG. 5 continues the schematic illustration of the process of biodiesel production as shown in FIG. 4, including demethylation and acidulation of crude glycerin.

FIG. 6 continues the schematic illustration of the process of biodiesel production in accordance with at least one embodiment of the present invention, as shown in FIG. 5, including crude glycerin and recycled glycerin storage.

FIG. 7 schematically illustrates a process in accordance with at least one embodiment of the present invention, including ion exclusion chromatography.

FIG. 8 schematically illustrates a process in accordance with at least one embodiment of the present invention, including crude glycerin polishing with anion and cation ion exchange resin.

FIG. 9 schematically illustrates a process in accordance with at least one embodiment of the present invention, including crude glycerin concentration through multiple stages of water evaporation, including the use of a multi-effect vacuum flash evaporator apparatus and crude glycerin water stripper apparatus.

FIG. 10 schematically illustrates a process in accordance with at least one embodiment of the present invention, including waste water desalination.

FIG. 11 graphically illustrates the separation of salts from transesterified crude glycerin via ion exclusion chromatography in accordance with at least one embodiment of the present invention.

FIG. 12 graphically illustrates a typical thermal process diagram according to Example 1.

FIG. 13 graphically illustrates a flow process diagram according to Examples 2 and 3.

FIG. 14 graphically illustrates a flow process diagram according to Example 4.

### DETAILED DESCRIPTION OF THE PREFERRED EMBODIMENTS

It shall be understood that as used throughout the specification and the claims crude glycerin, industrial grade glycerin, purified grade glycerin, recycled glycerin, glycerin product, and glycerin solution shall be understood to mean a solution comprising glycerol. Glycerol shall be understood to mean the chemical compound 1,2,3-Propanetriol.

It has now been found that in at least one embodiment of the present invention crude glycerin from sources such as biofuels production can be purified in a novel manner via the use of ion exclusion chromatography (“IEC”) for chromatographic separation fractionation in combination with glycerin solution dewatering/concentration. The use of IEC produces a glycerin solution in water that is substantially salt free. The glycerin solution product from ion exclusion chromatography can be concentrated by evaporating water to produce a valuable high volume petrochemical feedstock more economically than traditional crude glycerin purification processes.

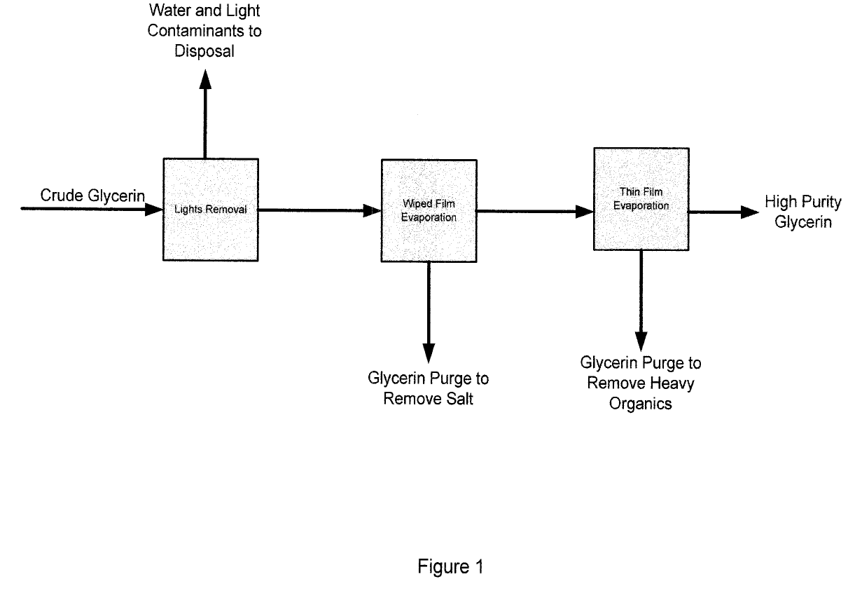

Referring to the drawings in FIG. 1, there is shown a broad overview of the traditional process for crude glycerin purification. The crude glycerin is first directed to a water and light contaminates removal step, which is then followed by a wiped film evaporation step, in which salts are removed, which is then followed by a thin film evaporation step in which heavy contaminants are removed. A high purity glycerin solution is thereby produced. However, as explained above, the traditional process involves the use of high temperatures, deep vacuums, and expensive process equipment.

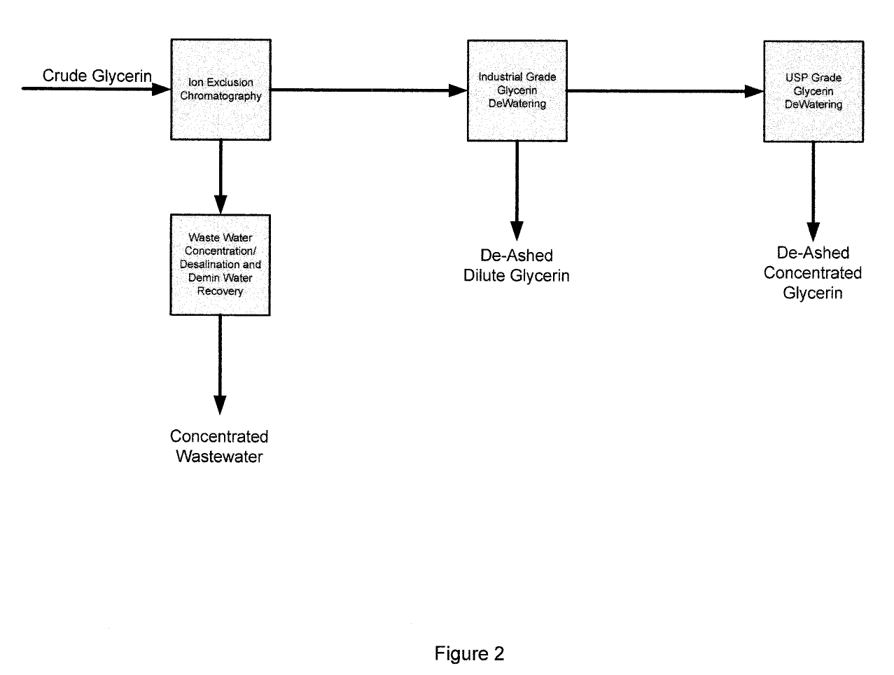

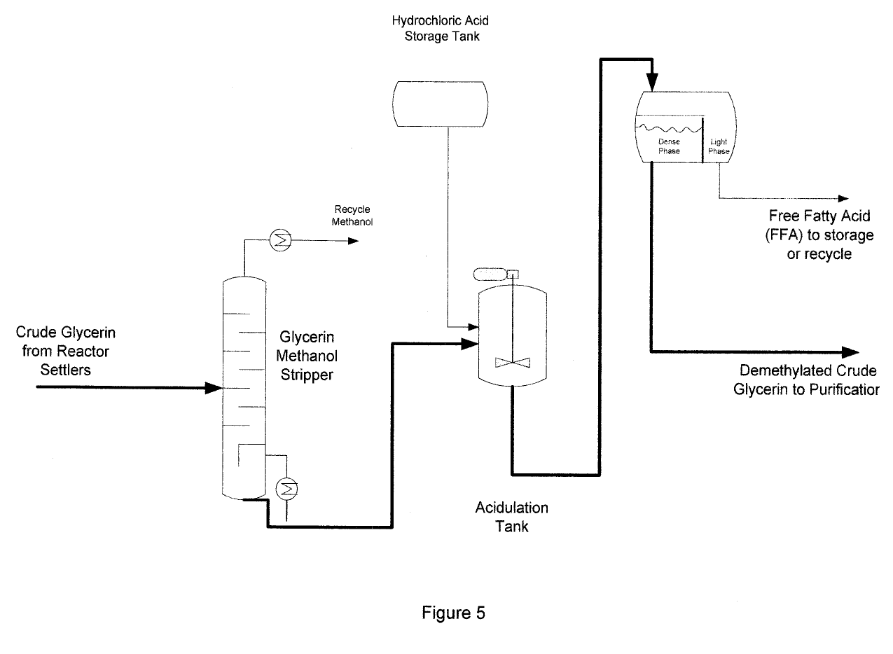

Referring now to FIG. 2, there is shown a broad overview of the features of the presently preferred embodiment of the invention. Crude glycerin is provided, for example as a by-product of biodiesel production, to an ion exclusion chromatography vessel capable of performing ion exclusion chromatography (EC) thereby allowing the fractionation of the crude glycerin. By way of example, the crude glycerin may be obtained from the generally known biodiesel production transesterification step, which is normally performed with the use of sodium methylate or potassium methylate catalysts. In the preferred embodiment said crude glycerin is provided from a crude glycerin storage tank which is upstream of the IEC step, as shown in FIG. 5. In addition to crude glycerin being obtained from biodiesel manufacturing, crude glycerin may also be obtained from other sources as for example from bioethanol still-bottoms and as a by-product of soap manufacturing.

The crude glycerin described above may comprise the following by-products: water, salts, MONG including free fatty acids (e.g., stearic acid and oleic acid), and fatty acid methyl esters (“FAME”). IEC utilizes a specific ion exchange resin designed for removing the by-products from the crude glycerin. For example, such ion exchange resins may include macroporous cation exchange resin such as Lewatit® GF303 available from LANXESS Deutschland GmbH. The ion exchange resin is more selective towards glycerol over the other crude glycerin by-products. Various methods for IEC are possible, for example, single column fixed bed processing, moving bed processing, or simulated moving bed processing may be used. In one embodiment, a single column fixed bed process is employed. Via the above mentioned ion exclusion chromatographic separation process the salts and other by-product of the crude glycerin are reduced.

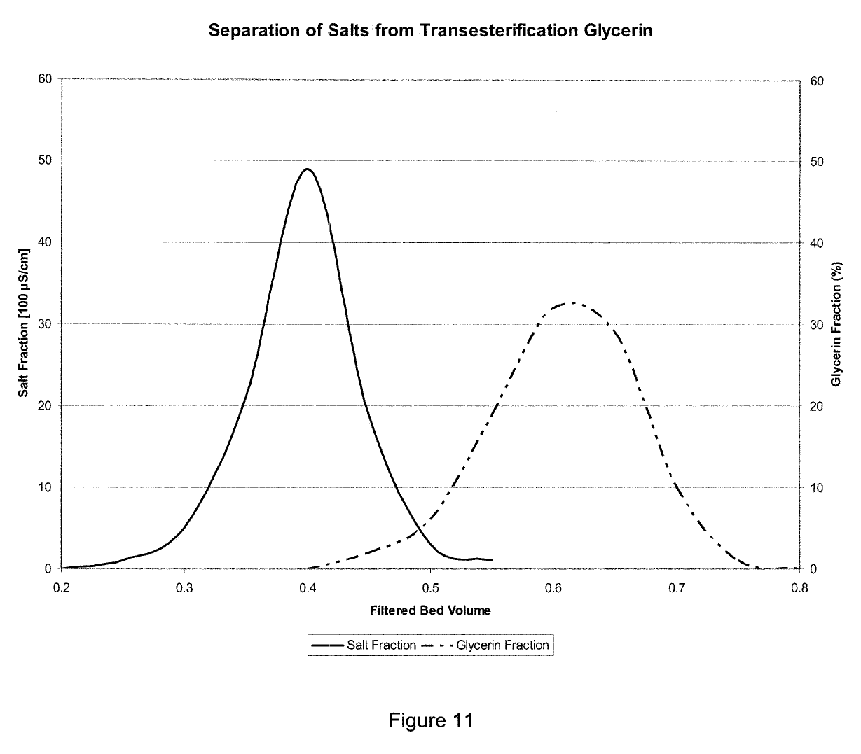

More specifically, in the preferred embodiment a pulsed amount of crude glycerin is provided to the ion exclusion vessel, the resin bed thereof is then washed with demineralized water provided from the demineralized water storage tank. The demineralized water first carries a majority of the crude glycerin by-products out of the bed leaving behind the majority of glycerol. Further flow of demineralized water then elutes the remaining glycerol with little residual salt contamination. A graph of the ion exclusion chromatographic separation of the crude glycerin is shown in FIG. 11.

The resin bed effluent is fractionated and monitored by refractive index and conductivity. Refractive index indicates the presence and concentration of glycerol in the fraction solution. Similarly, conductivity indicates the presence and concentration of ionic salts in the resin bed effluent. It will be appreciated that a number of fractions may be obtained via the IEC separation, which will then be further processed. In the preferred embodiment, up to four fractions of resin bed effluent are detected, segregated, and processed as described below. It should be appreciated that other fractionation monitoring and controlling methods may be employed, for example by means of on-line gas and/or ion chromatography or sequential events logic controllers.

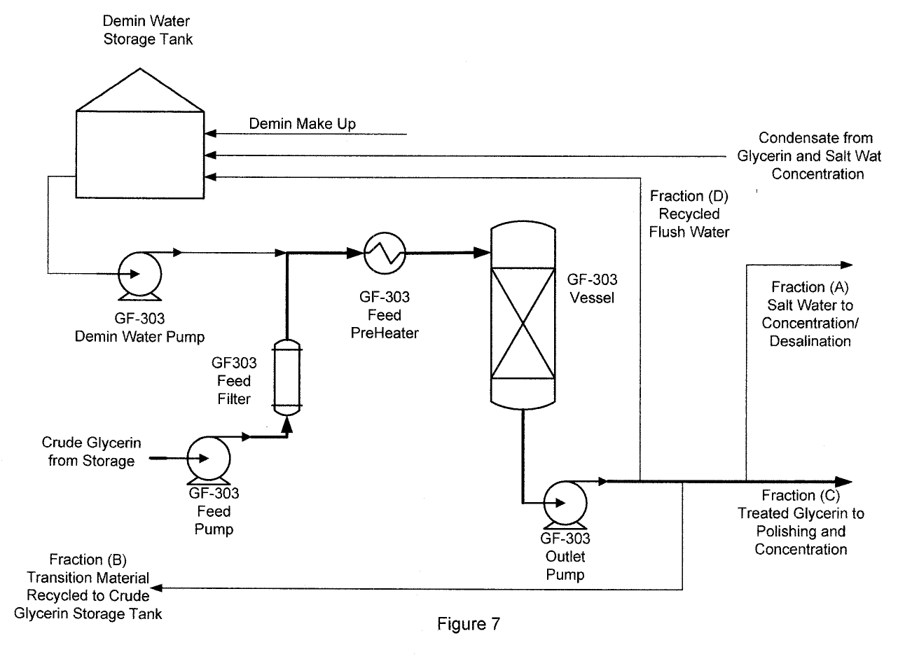

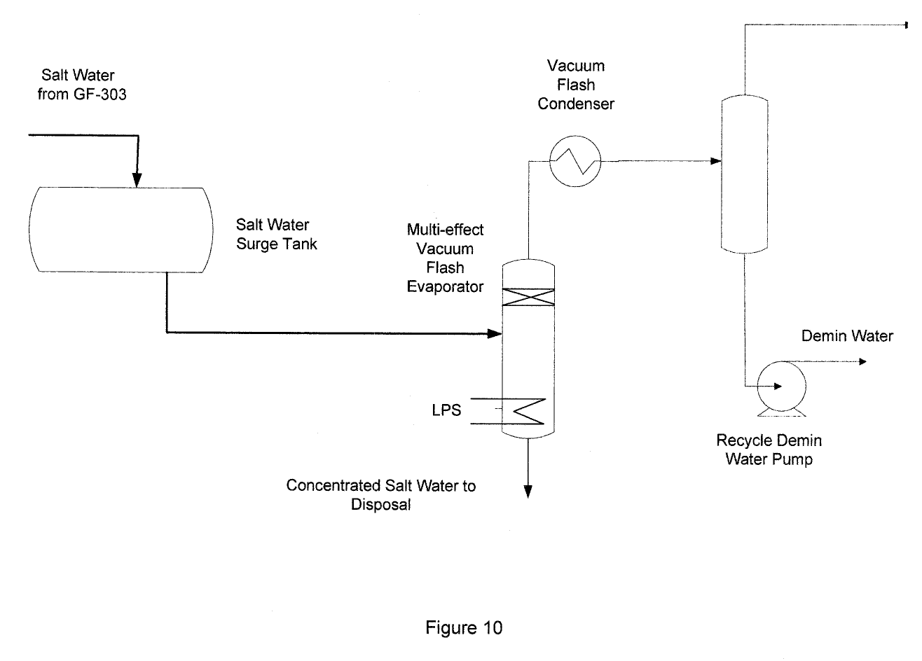

Various fractions can be obtained as part of process of the invention. As part of the preferred embodiment, a faction (A) may be collected, which comprises demineralized water with elevated salt content. This fraction may be characterized by high conductivity and a low refractive index. As shown in FIG. 2, this fraction is sent to waste water desalination then recycling and disposal. The concentration/desalination operation allows demineralized water to be recovered and recycled for re-use as shown in FIG. 10. As shown in FIG. 7, the demineralized water is eventually sent back to a demineralized water storage tank, which will then eventually return to the ion exclusion chromatography vessel. By concentrating the salt content of the waste stream and effectuating desalination, waste disposal costs may be reduced. The concentration/desalination of the salt water from the ion exclusion chromatography vessel includes providing the same to a multi-effect vacuum flash evaporator (hereinafter “MEVF evaporator”), which is also known as a multi-stage vacuum flash evaporator, which can drive off water (as liquid and/or vapor). The water driven off is, in turn, re-used in the IEC step as well. As can be appreciated by the skilled artisan, a vacuum flash condenser may be housed either within or outside of the MEVF evaporator.

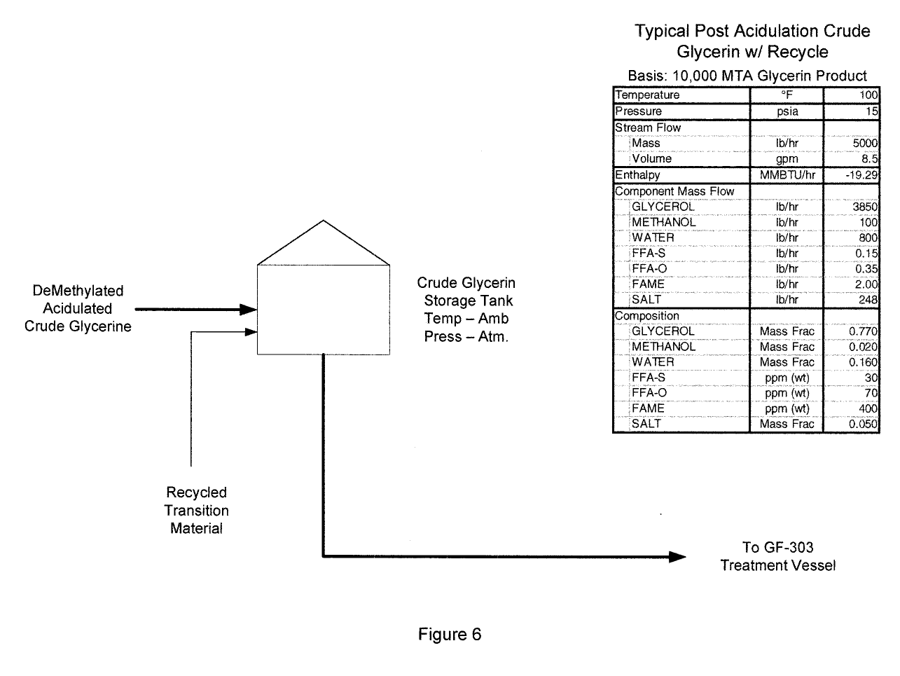

Further another fraction (B), according to the preferred embodiment, comprises demineralized water with small amounts of salt, other crude glycerin by-products, and glycerol. This fraction may be characterized as having reduced conductivity and measurable refractive index as compared to the preliminary fraction above. This fraction (B) can be recycled back to the crude glycerin storage, as shown in FIGS. 6 and 7, to allow recovery of the remaining glycerol contained in this fraction via the recirculation back into the crude glycerin solution stream entering the IEC separation step. It should be appreciated that if moving bed processing is utilized fraction (B) may be reduced or eliminated altogether from the process. The volume and existence of fraction (B) are dependent upon the ion exclusion chromatographic process employed and the details of its operation.

A further fraction (C), of the preferred embodiment, comprises demineralized water containing the majority of glycerol from the IEC separation process of the crude glycerin and has a significantly reduced amount of salt and other crude glycerin by-products as compared to fraction (B) and as can be appreciated with reference to the graph at FIG. 11. Fraction (C) is characterized by high refractive index and very low conductivity. In the preferred embodiment, this fraction is further processed to reduce the water from the solution as is shown in the figures and discussed below. Fraction (C) generally comprises about less than 100 ppm salts and crude glycerin by-products. The glycerol weight percent is about between 10 to 50 wt %.

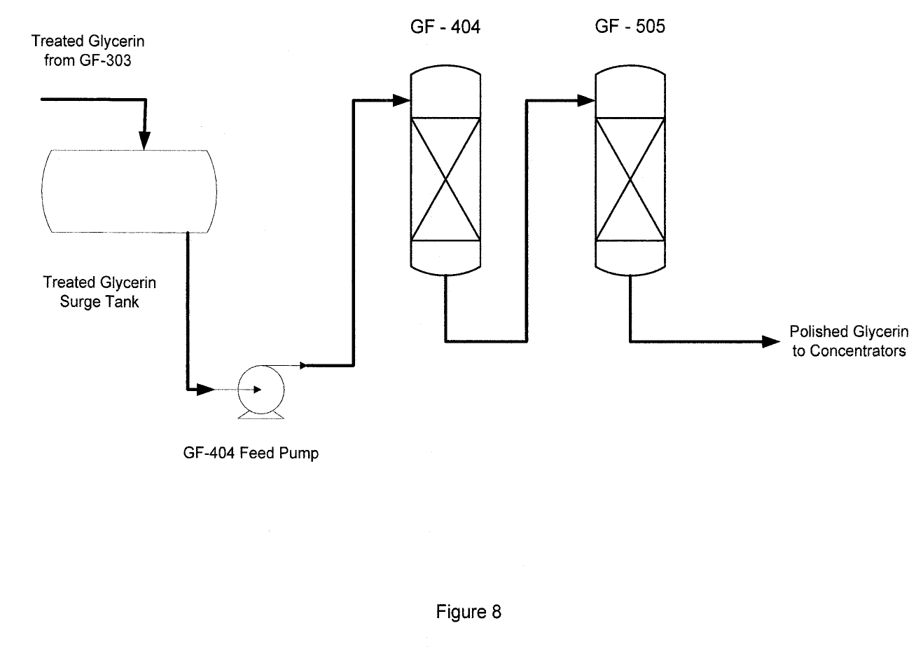

In one embodiment, as shown in FIG. 8, fraction (C) may undergo one or more additional intermediate ion exchange separation purification steps to thereby further purify the solution of salts and other by-products before proceeding onto the subsequent dewatering processing steps. The performance of the optional ion exchange purification step(s) can reduce the salts and other by-products from about 100 ppm to 1 ppm. The glycerol weight percent remains unchanged, about between 10 to 50 wt %. The additional ion exchange separation may include the use of one or more anion and/or cation ion exchange resins. For example such resins may include Lewatit® GF404 and GF505 available from LANXESS Deutschland GmbH.

As indicated above, in the preferred embodiment a fraction (D) may also be collected from the IEC process step (FIG. 7). Fraction (D) is comprised almost solely of demineralized water that may be recycled back to the demineralized water storage tank (FIG. 6) with no further processing, such as desalination/concentration, being required. Again, it should be appreciated that if moving bed processing is utilized fraction (D) may be reduced or eliminated altogether from the process. The volume and existence of fraction (D) are dependent upon the ion exclusion chromatographic process employed and the details of its operation.

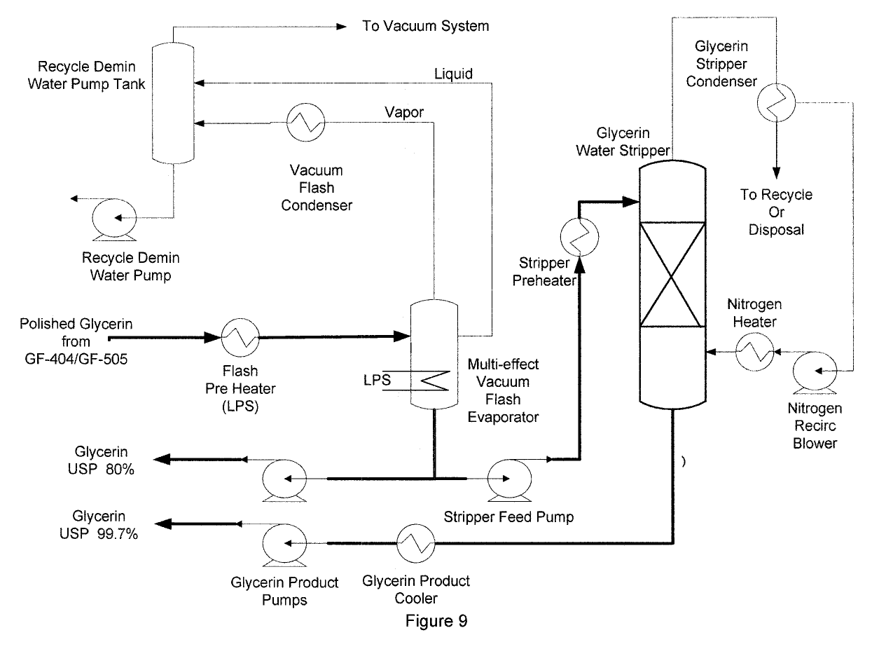

Fraction (C) of the preferred embodiment, whether being additionally purified or not, comprises glycerol and water, wherein the glycerol weight percent is about between 10 and 50 wt %. As illustrated in FIG. 2, an industrial grade glycerin solution dewatering step may be performed on fraction (C) by means of a multi-effect vacuum flash evaporator (MEVF) (FIG. 9), thereby removing water as liquid and/or vapor from fraction (C) by adding heat to vaporize a portion of the water. It should be understood that while various methods may be used to effectuate the removal of the water from fraction (C), for example by means of a single stage flash process, in the preferred embodiment use is made of a MEVF evaporator. The use of the MEVF evaporator as the means for the industrial grade glycerin solution dewatering step allows for the reduction of the heat required as compared to other water removal processes and, thereby, conserves energy and, in addition, reduces the amount of glycerol that is lost in the purged water stream.

As shown in FIG. 9, the purged water stream can be recycled back into the IEC step via a recycling demineralized water pump. An industrial grade glycerin solution product is produced from the MEVF evaporator process and can be collected. The industrial grade glycerin solution product comprises, in one embodiment, a glycerol weight percent between 60 to 95 wt % and in another embodiment about 75 to 85 wt %, and in another embodiment about 80 wt %. Salts and other crude glycerin by-products may be present as well, for example, in the amount of about 1 to 10 ppm, preferably about 1 to 5 ppm, and more preferably about 1 ppm.

The industrial grade glycerin solution product of the preferred embodiment is further concentrated to purified grade glycerin solution product by an additional water removal step. As shown in FIGS. 2 and 9, an industrial grade glycerin solution dewatering step by means of a glycerin water stripper apparatus is performed to produce a purified grade glycerin solution product. The industrial grade glycerin solution dewatering step of the present embodiment accomplishes the removal of additional water from the industrial grade glycerin solution by means of a recirculating nitrogen or air stream that strips the water from the industrial grade glycerin solution product at moderate temperatures and atmospheric or sub-atmospheric pressure, thereby, further increasing the efficiency of the process and reducing production costs.

In the preferred embodiment, the glycerin water stripper is a vertical pressure vessel with one or more beds of mass transfer packing to improve vapor liquid contact. Said pressure vessel may be divided into various areas such as, in ascending order, a bottom, middle, and top. Hot, dry recirculating nitrogen gas and/or air is introduced into the bottom of the glycerin water stripper. In one embodiment of the invention nitrogen gas is introduced. As the dry nitrogen flows upward through the packing and contacts the wet glycerin, the nitrogen is humidified with water from the glycerin solution and separated therefrom. The humidified nitrogen gas and water is then removed from the middle or top of the vessel. As the glycerin solution flows down through the packing, the water content continuously decreases. From the bottom of the vessel purified grade glycerin solution product is obtained and subsequently collected. It should be appreciated that the amount of water removal is a function of the design of the glycerin water stripper apparatus and can, therefore, be varied as desired.

In the preferred embodiment, the wet nitrogen leaves the glycerin water stripper through the top of the vessel and is sent to a glycerin stripper condenser to be cooled against air or cooling water (FIG. 9). This operation will also dehumidify the nitrogen stream. The water that condenses out of the recirculating nitrogen stream can be recycled to the demineralized water storage tank or sent to disposal. The dehumidified nitrogen from the condenser is then directed to a nitrogen recirculation blower to increase its pressure prior to being reintroduced into the glycerin water stripper. The operation of the nitrogen recirculation blower will cause the temperature of the nitrogen stream to rise slightly. If this increase is not adequate for the desired glycerin solution quality, however, a nitrogen heater can be utilized between the blower outlet and the glycerin water stripper inlet.

In the preferred embodiment, United States Pharmacopeia (USP) quality purified grade glycerin solution product is obtained from the purified grade glycerin solution dewatering step via the glycerin water stripper apparatus and has a glycerol weight percent of about between 95 to 100 wt % and in at least one embodiment about 99 wt %. In one embodiment, the purified grade glycerin solution product comprises less than 1 ppm salts and other crude glycerin by-products.

### EXAMPLES

To illustrate the current invention, examples of the purification of biodiesel derived crude glycerin are given below. The first comparative example describes the traditional thermal processes for the separation of glycerol from the major contaminants. The subsequent examples describe the processes according to the current invention.

The examples below were developed from computer models generated with the Aspen Plus steady state simulation software available from AspenTech. The NRTL property system within Aspen Plus was utilized to generate physical and thermodynamic properties. In all examples, a crude glycerin feed stream, typical of that from a biodiesel plant, was utilized and was defined to be 82 wt % glycerol, 7 wt % inorganic ash, 6 wt % MONG (matter organic, non glycerin), 4 wt % water and 1 wt % methanol. The target quality of refined glycerin was greater than 99.5 wt % glycerol, under 1000 wt ppm MONG, under 100 wt ppm inorganic ash and the balance being water.

### Example 1

### Comparative Thermal Process

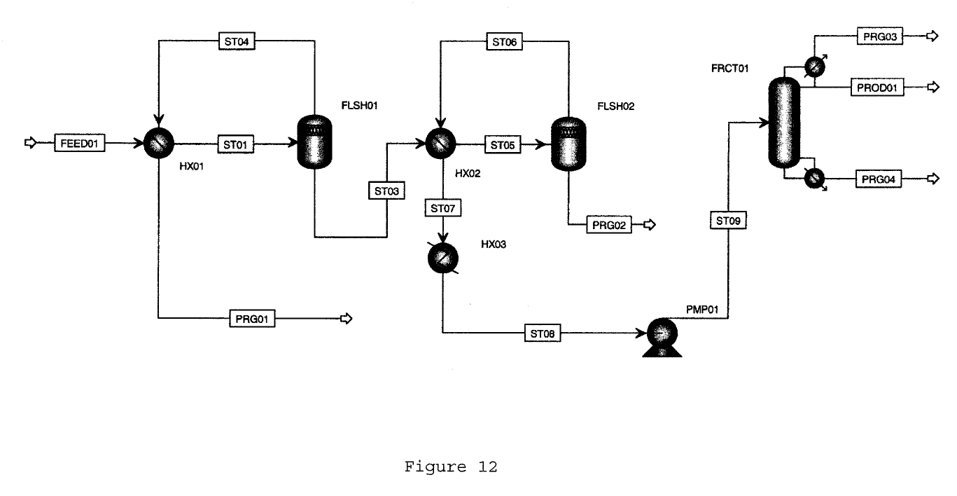

As depicted in FIG. 1, a traditional thermal process includes the following unit operations: (1) vaporization of methanol and water from the crude glycerin stream, (2) evaporation of glycerin and MONG from a salt waste stream and (3) evaporation of glycerin from MONG. Additional polishing steps are required to remove minor color and odor contaminants from the glycerin product. A process flow diagram for a typical thermal process with accompanying material and heat balance is given in FIG. 12 and Table 2, respectively.

With reference to FIG. 12, an incoming feed stream of crude glycerin is preheated to approaching 185° F. prior to being flashed into a separator FLSH01 operating under vacuum in a lights removal step. Heat from an external source is added to FLSH01 to drive methanol and water from the crude glycerin. The vapors generated can be condensed against the feed stream in heat exchanger HX01 to reduce heat requirements. In a single stage flash, the vapors generated will carry a small amount of glycerin with them contributing to an overall appreciable glycerin yield loss associated with the thermal process. Vapors generated in FLSH01 are condensed and form the first purge stream, PRG01.

The liquid stream from the lights removal FLSH01 step is further heated at heat exchanger HX02 and directed to a wiped film evaporator (WFE) modeled as FLSH02 to eliminate salts and other non-volatile contaminants. The WFE is an agitated thin film evaporator where the feed is introduced into the top of the evaporator and is spread into a thin film by rotating wiper blades as it flows down the conical sides of the evaporator. Vapor generation takes place as the thin film moves down the walls. As the remaining liquor thickens and becomes more viscous, the wiper blades direct the liquor to a bottom drain. Heat transfer area is limited to the walls and the heat transfer medium is usually high pressure steam or hot oil. Mechanical seals are required for the rotating shaft of the wiper blades which with the bearings of the shaft represent high maintenance components of the system. Depending upon the fluid nature of the bottoms salt purge, the bottoms flow will be controlled by either a flow control valve or, it the salt is sufficiently dry, a lock hopper or rotary valve assembly. The bottoms flow also carries with it appreciable amounts of glycerin that again contributes to the overall yield loss of the thermal process as well as amounts of high boiling compounds designated as MONG. This second purge stream is designated PRG02. The product as vapor generated from the WFE can be condensed in heat exchanger HX02 against the feed stream to reduce heat requirements.

The stabilized, de-ashed product from the WFE is sent to a high temperature, low pressure distillation column designated and modeled as FRCT01 to remove residual lights in an overhead purge stream designated PRG03 and MONG in a bottom draw purge stream designated PRG04 to produce a high quality glycerin product. Each of the two purge streams carries appreciable amounts of glycerol that further reduce purified glycerol yield.

In the Example 1 provided, the yield loss of contained glycerol is between 5% and 6% of the incoming crude glycerin. The net heat consumption is calculated to be 1170 BTU per pound of glycerol product.

### Example 2

### Fixed Bed with ME Evaporator and Water Stripper

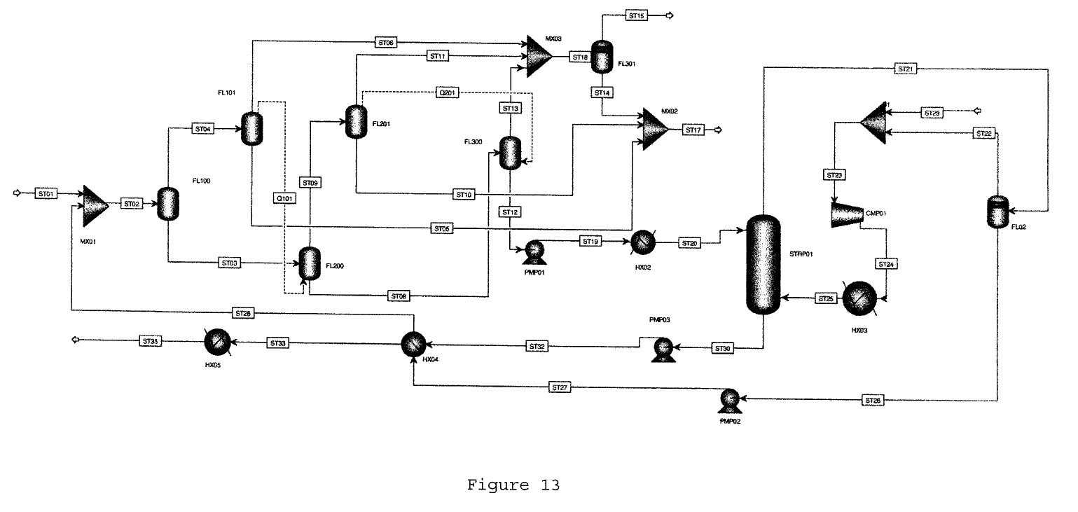

As depicted in FIGS. 6 through 9, the current invention teaches a process of purifying crude glycerin typically obtained as a by-product of biodiesel production. The process shown utilized ion exclusion chromatography combined with ion exchange to produce an intermediate glycerin product essentially devoid of all contaminants except water. The nature of the intermediate glycerin product allows utilization of a process that vaporizes water with mild operating conditions instead of attempting to boil glycerol to eliminate contamination. The water removal process taught by one embodiment of the present invention is depicted in FIG. 13, along with an accompanying material and heat balance provided in Table 3.

With reference to FIG. 13, from fixed bed ion exclusion chromatography, the intermediate glycerin product defined as fraction (C) in the detailed description of the invention and designated as ST01 in FIG. 13 will typically contain approximately 18 wt % glycerol in approximately 82 wt % water. Contaminants other than water can be expected to be in the parts per million range and will not effect final glycerol product quality. The use of fixed bed ion exclusion chromatography can be expected to result in a yield loss not to exceed 2%.

The first step of water removal utilizes a multiple effect evaporation process that utilizes successively decreasing pressure and temperature to make the optimal use of heat needed to vaporize water. Temperatures within the various sections of the multiple effect evaporator are maintained less than 240° F. to allow the use of low pressure steam. FIG. 13 shows a triple effect evaporator as flash and condensation devices with heat interchange. For convenience and accuracy, a triple effect evaporator is modeled as adiabatic flash evaporators FL100, FL200 and FL300. The condensing portion of the triple effect evaporator is modeled as FL101, FL201 and FL301. The heat generated in each of the condensing units is transferred to the corresponding flash evaporator through heat lines designated as Q101 and Q201. Use of triple effect evaporation is a single embodiment and should not be considered the only method of gross water removal. More or fewer effects can also be utilized depending on financial considerations and are considered to be within the scope of this invention. The intermediate glycerin product from multi-effect evaporation in this example is designated as ST12 and is 85 wt % glycerin. The vapor purge streams from the triple effect evaporator are designated ST06, ST11 and ST15 and are condensed in FL301. The liquid streams from FL101, FL201 and FL301 are combined and designated ST17. This purge stream is predominantly water with minor amounts of glycerin and methanol.

To reduce water content further, several means of fractionation can be considered. In this example of the current invention, a stripping column designated STRP01 operating at slightly above atmospheric pressure with suitable internals such as trays or packing is used to enable intimate contact between stripper feed stream ST20 and a re-circulating nitrogen stream that enters STRP01 as ST25. Stream ST20 enters the stripper through suitable designed liquid distribution equipment within the stripper and flows onto the top section of the column internals. Design of the mass transfer internals and liquid distribution equipment is left to those familiar with the art. Stream ST25 is a gaseous steam that enters the stripper below the column's internal mass transfer equipment. The re-circulating nitrogen stream ST25 is humidified predominantly with water and to a very small extent glycerol that is removed from the liquid feed stream ST20. The overhead vapor stream, designated ST21, from STRP01 is cooled and dehumidified in the combined heat exchanger and phase separator FL02. This unit may exist as either a single piece of equipment such as condenser with large liquid holdup or could be a condenser and separate liquid surge drum with liquid de-entrainment internals. The liquid generated from the dehumidification step is designated ST26 and is recycled to recover any glycerol that is vaporized in STRP01. The nitrogen recirculation stream ST22 from phase separator FL02 is compressed in blower CMP01 and heated against steam or another heat source in heat exchanger HX03. Product glycerin exits STRP01 as a bottom stream designated ST30 with less than 0.5 wt % water. The glycerin product stream may be cooled against various process streams to recover heat. In the example, heat is transferred from the glycerin product to the liquid recycle stream from FL02.

According to Example 2, operated in the manner taught in this invention, overall glycerol yield loss is less than 3%. The net heat consumption is calculated to be 2390 BTU per pound of glycerin product. This value includes a calculated conversion of electrical power consumed by CMP01 into a heat load.

It is readily apparent that glycerol recovery has been improved and energy utilization efficiency has been reduced. With respect to heat provided by steam, this aspect of the current invention can utilize low level steam to reduce costs relative to high pressure steam required for the thermal process. A financial analysis may show that using greater amounts of low level waste heat can be more cost effective than expensive high pressure steam required in the traditional purification process described in Example 1.

### Example 3

### Simulated Moving Bed with ME Evaporator and Water Stripper

Although a fixed bed operation of ion exclusion chromatography (IEC) will produce the required product quality, it is readily apparent that the water content of the intermediate glycerin product stream is great and contributing to high energy consumption. Fortunately, there exists technology that raises the operating efficiency of EC with respect to water usage and energy consumption.

Traditionally, continuous operations impart a degree of efficiency that batch operations can not. The efficiency of fixed bed chromatography could be improved by converting to a continuous mode of operation with fluids flowing in one direction and the ion exclusion resin flowing counter current to the liquid. In practice, this is impractical, if not essentially impossible. But this mode of operation can be approached by having liquids flowing continually and simulating the movement of the bed, which actually remains stationary. Such technology, designated Simulated Moving Bed (SMB) may therefore be utilized in another embodiment of the present invention.

For glycerin purification the use of SMB along with ion exchange will produce a dilute glycerin product similar to the fixed bed process previously described with the exception of having a much lower water content. Whereas the glycerin effluent of the fixed bed EC was 82 wt % water, the water content from SMB, in comparison, is only 46.4 wt % water for this example. Water content of the glycerin product leaving the SMB segment of the process may be varied depending upon technical and economic considerations. The exact water content of the glycerin product leaving the SMB segment does not affect the lessons taught in the present invention.

The unit operations for the dewatering system for this Example 3 are identical to those of Example 2. This Example 3 can utilize the process flow shown in FIG. 13. The material and heat balance is provided in Table 4.

Operations according to Example 3, result in an overall glycerol yield loss of less than 3%. The net heat consumption as a result of using SMB is calculated to be 824 BTU per pound of glycerin product. This provides a great improvement over Examples 1 and 2. Additionally, the process equipment required for water removal in Example 3 will be substantially smaller and less expensive than the comparable equipment in Example 2

### Example 4

### Simulated Moving Bed with ME Evaporator and Drying Column

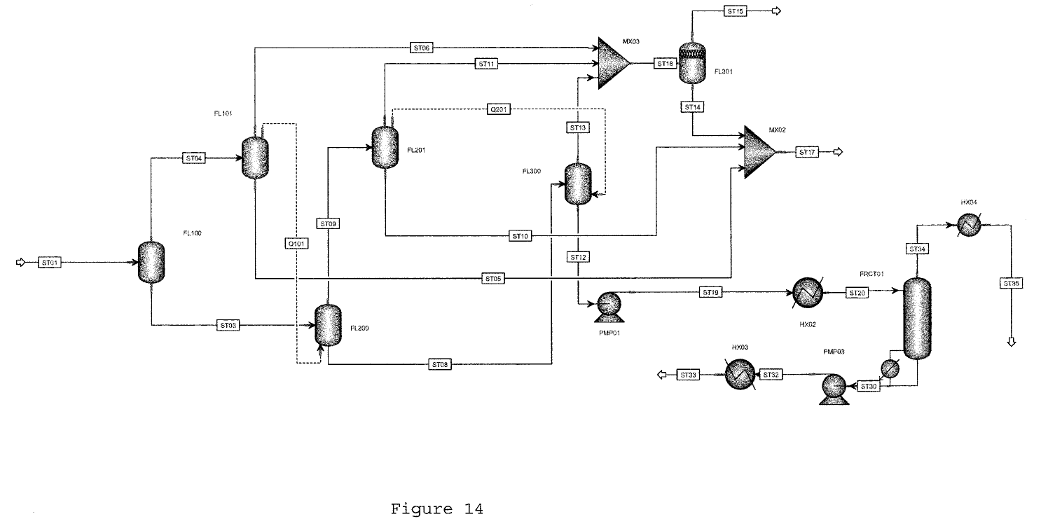

The use of a re-circulating nitrogen stream to strip water from wet glycerin takes advantage of low level waste heat. If higher pressure steam is available and economics can justify its use, the stripping column and its auxiliary heat exchangers and compressors can be replaced by a vacuum drying column that utilizes a reboiler and condenser. One embodiment of the water removal process according to the invention is depicted in FIG. 14 and a material and heat balance is given in Table 5.

Multi effect evaporation is used to reduce the water content of the process glycerin stream from 46.4 wt % to 85 wt % in the same manner as Example 3.

In the present embodiment there is utilized either a tray or packed column with heat exchangers for preheat, reboil and condensation. The fractionation column, instead of operating at slightly above atmospheric pressure, requires vacuum conditions to keep temperatures in a more moderate range. Even with substantial vacuum, the process side of the reboiler must operate over 300° F. which is 70° F. hotter than the bottom of the stripping column in Example 3.

Operated in accordance with Example 4, the overall glycerol yield loss in is less than 3%. The net heat consumption is calculated to be 753 BTU per pound of glycerol product. This provides some improvement over Example 3 but requires higher pressure steam for the reboiler and preheater.

### Tables:

TABLE 1
Comparison of the various Examples

| | | | Net Energy |
|---|---|---|---|
| | | Glycerin | Consumption |
| | Description | Recovery | (BTU/lb product) |
| 1 | Thermal Process | 94% | 1170 |
| 2 | Fixed Bed with ME Evaporator and | 97% | 2390 |
| | Water Stripper | | |
| 3 | SMB with ME Evaporator | 97% | 824 |
| | and Water Stripper | | |
| 4 | SMB with ME Evaporator | 97% | 753 |
| | and Drying Column | | |

TABLE 2

| | FEED01 | PRG01 | PRG02 | PRG03 | PRG04 | PROD01 | ST01 | ST03 |
|---|---|---|---|---|---|---|---|---|
| Temperature F. | 100.00 | 105.00 | 407.00 | 274.40 | 378.90 | 274.40 | 184.90 | 294.40 |
| Pressure psi | 50.00 | 2.50 | 0.10 | 0.20 | 0.40 | 0.20 | 50.00 | 2.50 |
| Vapor Frac | 0.000 | 0.000 | 0.000 | 1.000 | 0.000 | 0.000 | 0.000 | 0.000 |
| Mole Flow lbmol/hr | 45.597 | 7.753 | 4.725 | 1.577 | 1.576 | 29.965 | 45.597 | 37.844 |
| Mass Flow lb/hr | 3550.000 | 166.828 | 310.530 | 49.992 | 264.763 | 2757.887 | 3550.000 | 3383.172 |
| Volume Flow cuft/hr | 44.919 | 2.759 | 3.220 | 62115.430 | 5.279 | 37.456 | 46.021 | 44.735 |
| Enthalpy MMBtu/hr | −11.027 | −0.956 | −0.763 | −0.199 | −0.454 | −8.286 | −10.869 | −9.721 |
| Mass Flow lb/hr | | | | | | | | |
| GLYCEROL | 2911.0000 | 15.7180 | 34.7580 | 25.5020 | 88.1660 | 2746.8550 | 2911.0000 | 2895.2820 |
| METHANOL | 35.5000 | 33.0150 | 0.0000 | 2.4110 | 0.0000 | 0.0730 | 35.5000 | 2.4850 |
| WATER | 142.0000 | 118.0310 | 0.0010 | 22.0670 | 0.0000 | 1.9010 | 142.0000 | 23.9690 |
| MONG-1 | 88.7500 | 0.0120 | 14.0890 | 0.0000 | 74.5750 | 0.0750 | 88.7500 | 88.7380 |
| MONG-2 | 88.7500 | 0.0220 | 11.1650 | 0.0000 | 77.1310 | 0.4320 | 88.7500 | 88.7280 |
| MONG-3 | 35.5000 | 0.0300 | 2.0170 | 0.0110 | 24.8910 | 8.5510 | 35.5000 | 35.4700 |
| ASH | 248.5000 | 0.0000 | 248.5000 | 0.0000 | 0.0000 | 0.0000 | 248.5000 | 248.5000 |
| Mass Frac | | | | | | | | |
| GLYCEROL | 0.8200 | 0.0940 | 0.1120 | 0.5100 | 0.3330 | 0.9960 | 0.8200 | 0.8560 |
| METHANOL | 0.0100 | 0.1980 | 0.0000 | 0.0480 | 0.0000 | 0.0000 | 0.0100 | 0.0010 |
| WATER | 0.0400 | 0.7080 | 0.0000 | 0.4410 | 0.0000 | 0.0010 | 0.0400 | 0.0070 |
| MONG-1 | 0.0250 | 0.0000 | 0.0450 | 0.0000 | 0.2820 | 0.0000 | 0.0250 | 0.0260 |
| MONG-2 | 0.0250 | 0.0000 | 0.0360 | 0.0000 | 0.2910 | 0.0000 | 0.0250 | 0.0260 |
| MONG-3 | 0.0100 | 0.0000 | 0.0060 | 0.0000 | 0.0940 | 0.0030 | 0.0100 | 0.0100 |
| ASH | 0.0700 | 0.0000 | 0.8000 | 0.0000 | 0.0000 | 0.0000 | 0.0700 | 0.0730 |

| | ST04 | ST05 | ST06 | ST07 | ST08 | ST09 |
|---|---|---|---|---|---|---|
| Temperature F. | 294.40 | 334.20 | 407.00 | 335.00 | 116.70 | 116.80 |
| Pressure psi | 2.50 | 2.50 | 0.10 | 0.10 | 0.09 | 5.00 |
| Vapor Frac | 1.000 | 0.018 | 1.000 | 1.000 | 0.000 | 0.000 |
| Mole Flow lbmol/hr | 7.753 | 37.844 | 33.118 | 33.118 | 33.118 | 33.118 |
| Mass Flow lb/hr | 166.828 | 3383.172 | 3072.642 | 3072.642 | 3072.642 | 3072.642 |
| Volume Flow cuft/hr | 25071.795 | 2327.396 | 3079880.0 | 2823790.0 | 40.850 | 40.851 |
| Enthalpy MMBtu/hr | −0.797 | −9.631 | −7.695 | −7.785 | −9.258 | −9.258 |
| Mass Flow lb/hr | | | | | | |
| GLYCEROL | 15.7180 | 2895.2820 | 2860.5240 | 2860.5240 | 2860.5240 | 2860.5240 |
| METHANOL | 33.0150 | 2.4850 | 2.4840 | 2.4840 | 2.4840 | 2.4840 |
| WATER | 118.0310 | 23.9690 | 23.9680 | 23.9680 | 23.9680 | 23.9680 |
| MONG-1 | 0.0120 | 88.7380 | 74.6490 | 74.6490 | 74.6490 | 74.6490 |
| MONG-2 | 0.0220 | 88.7280 | 77.5630 | 77.5630 | 77.5630 | 77.5630 |
| MONG-3 | 0.0300 | 35.4700 | 33.4530 | 33.4530 | 33.4530 | 33.4530 |
| ASH | 0.0000 | 248.5000 | 0.0000 | 0.0000 | 0.0000 | 0.0000 |
| Mass Frac | | | | | | |
| GLYCEROL | 0.0940 | 0.8560 | 0.9310 | 0.9310 | 0.9310 | 0.9310 |
| METHANOL | 0.1980 | 0.0010 | 0.0010 | 0.0010 | 0.0010 | 0.0010 |
| WATER | 0.7080 | 0.0070 | 0.0080 | 0.0080 | 0.0080 | 0.0080 |
| MONG-1 | 0.0000 | 0.0260 | 0.0240 | 0.0240 | 0.0240 | 0.0240 |
| MONG-2 | 0.0000 | 0.0260 | 0.0250 | 0.0250 | 0.0250 | 0.0250 |
| MONG-3 | 0.0000 | 0.0100 | 0.0110 | 0.0110 | 0.0110 | 0.0110 |
| ASH | 0.0000 | 0.0730 | 0.0000 | 0.0000 | 0.0000 | 0.0000 |

TABLE 3

| | ST01 | ST02 | ST03 | ST04 | ST05 | ST06 | ST08 | ST09 |
|---|---|---|---|---|---|---|---|---|
| Temperature F. | 100.00 | 103.60 | 227.50 | 227.50 | 222.70 | 222.70 | 207.80 | 207.80 |
| Pressure psi | 25.00 | 25.00 | 18.00 | 18.00 | 17.00 | 17.00 | 12.00 | 12.00 |
| Vapor Frac | 0.000 | 0.000 | 0.000 | 1.000 | 0.000 | 1.000 | 0.000 | 1.000 |
| Mole Flow lbmol/hr | 744.119 | 770.823 | 545.863 | 224.960 | 224.937 | 0.022 | 308.513 | 237.351 |
| Mass Flow lb/hr | 15675.000 | 16160.906 | 12103.588 | 4057.319 | 4056.913 | 0.406 | 7823.066 | 4280.522 |
| Volume Flow cuft/hr | 244.191 | 252.600 | 199.811 | 91235.487 | 71.268 | 9.596 | 122.709 | 140648.943 |
| Enthalpy MMBtu/hr | −96.288 | −99.531 | −70.413 | −23.130 | −27.192 | −0.002 | −41.908 | −24.439 |
| Mass Flow lb/hr | | | | | | | | |
| GLYCEROL | 2820.0000 | 2826.0000 | 2820.6820 | 5.3180 | 5.3180 | 0.0000 | 2815.0960 | 5.5870 |
| WATER | 12853.0000 | 13332.9050 | 9281.6610 | 4051.2440 | 4050.8400 | 0.4050 | 5006.9310 | 4274.7300 |
| NACL | 1.0000 | 1.0000 | 1.0000 | 0.0000 | 0.0000 | 0.0000 | 1.0000 | 0.0000 |
| METHANOL | 1.0000 | 1.0010 | 0.2440 | 0.7570 | 0.7560 | 0.0010 | 0.0390 | 0.2050 |
| NITROGEN | 0.0000 | 0.0000 | 0.0000 | 0.0000 | 0.0000 | 0.0000 | 0.0000 | 0.0000 |
| Mass Frac | | | | | | | | |
| GLYCEROL | 0.1800 | 0.1750 | 0.2330 | 0.0010 | 0.0010 | 0.0000 | 0.3600 | 0.0010 |
| WATER | 0.8200 | 0.8250 | 0.7670 | 0.9990 | 0.9990 | 0.9980 | 0.6400 | 0.9990 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0020 | 0.0000 | 0.0000 |
| NITROGEN | 0.0000 | 0.0000 | 0.0000 | 0.0000 | 0.0000 | 0.0000 | 0.0000 | 0.0000 |
| Mole Flow lbmol/hr | | | | | | | | |
| GLYCEROL | 30.6210 | 30.6860 | 30.6280 | 0.0580 | 0.0580 | 0.0000 | 30.5670 | 0.0610 |
| WATER | 713.4500 | 740.0890 | 515.2100 | 224.8780 | 224.8560 | 0.0220 | 277.9270 | 237.2840 |
| NACL | 0.0170 | 0.0170 | 0.0170 | 0.0000 | 0.0000 | 0.0000 | 0.0170 | 0.0000 |
| METHANOL | 0.0310 | 0.0310 | 0.0080 | 0.0240 | 0.0240 | 0.0000 | 0.0010 | 0.0060 |
| NITROGEN | 0.0000 | 0.0000 | 0.0000 | 0.0000 | 0.0000 | 0.0000 | 0.0000 | 0.0000 |
| Mole Frac | | | | | | | | |
| GLYCEROL | 0.0410 | 0.0400 | 0.0560 | 0.0000 | 0.0000 | 0.0000 | 0.0990 | 0.0000 |
| WATER | 0.9590 | 0.9600 | 0.9440 | 1.0000 | 1.0000 | 0.9990 | 0.9010 | 1.0000 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0010 | 0.0000 | 0.0000 |
| NITROGEN | 0.0000 | 0.0000 | 0.0000 | 0.0000 | 0.0000 | 0.0000 | 0.0000 | 0.0000 |

| | ST10 | ST11 | ST12 | ST13 | ST14 | ST15 | ST17 | ST18 |
|---|---|---|---|---|---|---|---|---|
| Temperature F. | 201.40 | 201.40 | 188.80 | 188.80 | 166.60 | 166.60 | 166.60 | 188.80 |
| Pressure psi | 11.00 | 11.00 | 6.00 | 6.00 | 5.00 | 5.00 | 5.00 | 6.00 |
| Vapor Frac | 0.000 | 1.000 | 0.000 | 1.000 | 0.000 | 1.000 | 0.031 | 1.000 |
| Mole Flow lbmol/hr | 237.327 | 0.024 | 57.921 | 250.592 | 250.613 | 0.025 | 712.877 | 250.638 |
| Mass Flow lb/hr | 4280.094 | 0.428 | 3299.766 | 4523.300 | 4523.682 | 0.452 | 12860.689 | 4524.134 |
| Volume Flow cuft/hr | 74.170 | 15.204 | 44.080 | 289511.305 | 76.704 | 33.570 | 29681.044 | 289566.408 |
| Enthalpy MMBtu/hr | −28.790 | −0.002 | −11.709 | −25.846 | −30.582 | −0.003 | −86.564 | −25.851 |
| Mass Flow lb/hr | | | | | | | | |
| GLYCEROL | 5.5870 | 0.0000 | 2804.1520 | 10.9440 | 10.9440 | 0.0000 | 21.8480 | 10.9440 |
| WATER | 4274.3030 | 0.4270 | 494.6130 | 4512.3180 | 4512.6990 | 0.4510 | 12837.8410 | 4513.1500 |
| NACL | 0.0000 | 0.0000 | 1.0000 | 0.0000 | 0.0000 | 0.0000 | 0.0000 | 0.0000 |
| METHANOL | 0.2050 | 0.0000 | 0.0010 | 0.0380 | 0.0390 | 0.0000 | 1.0000 | 0.0390 |
| NITROGEN | 0.0000 | 0.0000 | 0.0000 | 0.0000 | 0.0000 | 0.0000 | 0.0000 | 0.0000 |
| Mass Frac | | | | | | | | |
| GLYCEROL | 0.0010 | 0.0000 | 0.8500 | 0.0020 | 0.0020 | 0.0000 | 0.0020 | 0.0020 |
| WATER | 0.9990 | 0.9990 | 0.1500 | 0.9980 | 0.9980 | 1.0000 | 0.9980 | 0.9980 |
| NACL | 0.0000 | 0.0000 | 0.0000 | 0.0000 | 0.0000 | 0.0000 | 0.0000 | 0.0000 |
| METHANOL | 0.0000 | 0.0010 | 0.0000 | 0.0000 | 0.0000 | 0.0000 | 0.0000 | 0.0000 |
| NITROGEN | 0.0000 | 0.0000 | 0.0000 | 0.0000 | 0.0000 | 0.0000 | 0.0000 | 0.0000 |
| Mole Flow lbmol/hr | | | | | | | | |
| GLYCEROL | 0.0610 | 0.0000 | 30.4490 | 0.1190 | 0.1190 | 0.0000 | 0.2370 | 0.1190 |
| WATER | 237.2600 | 0.0240 | 27.4550 | 250.4720 | 250.4930 | 0.0250 | 712.6080 | 250.5180 |
| NACL | 0.0000 | 0.0000 | 0.0170 | 0.0000 | 0.0000 | 0.0000 | 0.0000 | 0.0000 |
| METHANOL | 0.0060 | 0.0000 | 0.0000 | 0.0010 | 0.0010 | 0.0000 | 0.0310 | 0.0010 |
| NITROGEN | 0.0000 | 0.0000 | 0.0000 | 0.0000 | 0.0000 | 0.0000 | 0.0000 | 0.0000 |
| Mole Frac | | | | | | | | |
| GLYCEROL | 0.0000 | 0.0000 | 0.5260 | 0.0000 | 0.0000 | 0.0000 | 0.0000 | 0.0000 |
| WATER | 1.0000 | 1.0000 | 0.4740 | 1.0000 | 1.0000 | 1.0000 | 1.0000 | 1.0000 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.0000 | 0.0000 | 0.0000 | 0.0000 | 0.0000 | 0.0000 | 0.0000 |

| | ST19 | ST20 | ST21 | ST22 | ST23 | ST24 | ST25 | ST26 |
|---|---|---|---|---|---|---|---|---|
| Temperature F. | 188.80 | 275.00 | 170.80 | 100.00 | 100.00 | 225.00 | 250.00 | 100.00 |
| Pressure psi | 30.00 | 25.00 | 16.00 | 15.00 | 15.00 | 27.50 | 26.50 | 15.00 |
| Vapor Frac | 0.000 | 0.081 | 1.000 | 1.000 | 1.000 | 1.000 | 1.000 | 0.000 |
| Mole Flow lbmol/hr | 57.921 | 57.921 | 499.837 | 473.133 | 473.115 | 473.115 | 473.115 | 26.704 |
| Mass Flow lb/hr | 3299.766 | 3299.766 | 13486.204 | 13000.298 | 13000.000 | 13000.000 | 13000.000 | 485.906 |
| Volume Flow cuft/hr | 44.080 | 1515.013 | 211224.591 | 189315.187 | 189299.949 | 126375.131 | 135953.985 | 7.909 |
| Enthalpy MMBtu/hr | −11.709 | −11.464 | −5.095 | −2.565 | −2.563 | −2.148 | −2.064 | −3.301 |
| Mass Flow lb/hr | | | | | | | | |
| GLYCEROL | 2804.1520 | 2804.1520 | 6.0270 | 0.0270 | 0.0270 | 0.0270 | 0.0270 | 6.0000 |
| WATER | 494.6130 | 494.6130 | 937.2680 | 457.3630 | 457.0010 | 457.0010 | 457.0010 | 479.9050 |
| NACL | 1.0000 | 1.0000 | 0.0000 | 0.0000 | 0.0000 | 0.0000 | 0.0000 | 0.0000 |
| METHANOL | 0.0010 | 0.0010 | 0.0220 | 0.0200 | 0.0200 | 0.0200 | 0.0200 | 0.0010 |
| NITROGEN | 0.0000 | 0.0000 | 12542.8870 | 12542.8870 | 12542.9510 | 12542.9510 | 12542.9510 | 0.0000 |
| Mass Frac | | | | | | | | |
| GLYCEROL | 0.8500 | 0.8500 | 0.0000 | 0.0000 | 0.0000 | 0.0000 | 0.0000 | 0.0120 |
| WATER | 0.1500 | 0.1500 | 0.0690 | 0.0350 | 0.0350 | 0.0350 | 0.0350 | 0.9880 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.0000 | 0.9300 | 0.9650 | 0.9650 | 0.9650 | 0.9650 | 0.0000 |
| Mole Flow lbmol/hr | | | | | | | | |
| GLYCEROL | 30.4490 | 30.4490 | 0.0650 | 0.0000 | 0.0000 | 0.0000 | 0.0000 | 0.0650 |
| WATER | 27.4550 | 27.4550 | 52.0260 | 25.3870 | 25.3670 | 25.3670 | 25.3670 | 26.6390 |
| NACL | 0.0170 | 0.0170 | 0.0000 | 0.0000 | 0.0000 | 0.0000 | 0.0000 | 0.0000 |
| METHANOL | 0.0000 | 0.0000 | 0.0010 | 0.0010 | 0.0010 | 0.0010 | 0.0010 | 0.0000 |
| NITROGEN | 0.0000 | 0.0000 | 447.7450 | 447.7450 | 447.7470 | 447.7470 | 447.7470 | 0.0000 |
| Mole Frac | | | | | | | | |
| GLYCEROL | 0.5260 | 0.5260 | 0.0000 | 0.0000 | 0.0000 | 0.0000 | 0.0000 | 0.0020 |
| WATER | 0.4740 | 0.4740 | 0.1040 | 0.0540 | 0.0540 | 0.0540 | 0.0540 | 0.9980 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.0000 | 0.8960 | 0.9460 | 0.9460 | 0.9460 | 0.9460 | 0.0000 |
| | ST27 | ST28 | ST29 | ST30 | ST32 | ST33 | ST35 | ST40 |
| Temperature F. | 100.10 | 210.60 | 100.00 | 235.50 | 235.60 | 195.70 | 100.00 | 100.00 |
| Pressure psi | 25.00 | 25.00 | 25.00 | 16.50 | 20.00 | 20.00 | 18.00 | 15.00 |
| Vapor Frac | 0.000 | 0.000 | 1.000 | 0.000 | 0.000 | 0.000 | 0.000 | 1.000 |
| Mole Flow lbmol/hr | 26.704 | 26.704 | 0.357 | 31.199 | 31.199 | 31.199 | 31.199 | 0.375 |
| Mass Flow lb/hr | 485.906 | 485.906 | 10.000 | 2813.562 | 2813.562 | 2813.562 | 2813.562 | 10.298 |
| Volume Flow cuft/hr | 7.909 | 8.445 | 85.715 | 37.515 | 37.516 | 36.941 | 35.930 | 149.949 |
| Enthalpy MMBtu/hr | −3.301 | −3.243 | 0.000 | −8.433 | −8.432 | −8.490 | −8.624 | −0.002 |
| Mass Flow lb/hr | | | | | | | | |
| GLYCEROL | 6.0000 | 6.0000 | 0.0000 | 2798.1520 | 2798.1520 | 2798.1520 | 2798.1520 | 0.0000 |
| WATER | 479.9050 | 479.9050 | 0.0000 | 14.3450 | 14.3450 | 14.3450 | 14.3450 | 0.3620 |
| NACL | 0.0000 | 0.0000 | 0.0000 | 1.0000 | 1.0000 | 1.0000 | 1.0000 | 0.0000 |
| METHANOL | 0.0010 | 0.0010 | 0.0000 | 0.0000 | 0.0000 | 0.0000 | 0.0000 | 0.0000 |
| NITROGEN | 0.0000 | 0.0000 | 10.0000 | 0.0640 | 0.0640 | 0.0640 | 0.0640 | 9.9360 |
| Mass Frac | | | | | | | | |
| GLYCEROL | 0.0120 | 0.0120 | 0.0000 | 0.9950 | 0.9950 | 0.9950 | 0.9950 | 0.0000 |
| WATER | 0.9880 | 0.9880 | 0.0000 | 0.0050 | 0.0050 | 0.0050 | 0.0050 | 0.0350 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.0000 | 1.0000 | 0.0000 | 0.0000 | 0.0000 | 0.0000 | 0.9650 |
| Mole Flow lbmol/hr | | | | | | | | |
| GLYCEROL | 0.0650 | 0.0650 | 0.0000 | 30.3830 | 30.3830 | 30.3830 | 30.3830 | 0.0000 |
| WATER | 26.6390 | 26.6390 | 0.0000 | 0.7960 | 0.7960 | 0.7960 | 0.7960 | 0.0200 |
| NACL | 0.0000 | 0.0000 | 0.0000 | 0.0170 | 0.0170 | 0.0170 | 0.0170 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.0000 | 0.3570 | 0.0020 | 0.0020 | 0.0020 | 0.0020 | 0.3550 |
| Mole Frac | | | | | | | | |
| GLYCEROL | 0.0020 | 0.0020 | 0.0000 | 0.9740 | 0.9740 | 0.9740 | 0.9740 | 0.0000 |
| WATER | 0.9980 | 0.9980 | 0.0000 | 0.0260 | 0.0260 | 0.0260 | 0.0260 | 0.0540 |
| NACL | 0.0000 | 0.0000 | 0.0000 | 0.0010 | 0.0010 | 0.0010 | 0.0010 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.0000 | 1.0000 | 0.0000 | 0.0000 | 0.0000 | 0.0000 | 0.9460 |

TABLE 4

| | ST01 | ST02 | ST03 | ST04 | ST05 | ST06 | ST08 | ST09 | ST10 |
|---|---|---|---|---|---|---|---|---|---|
| Temperature F. | 100.00 | 109.20 | 229.30 | 229.30 | 222.50 | 222.50 | 210.00 | 210.00 | 201.40 |
| Pressure psi | 25.00 | 25.00 | 18.00 | 18.00 | 17.00 | 17.00 | 12.00 | 12.00 | 11.00 |
| Vapor Frac | 0.000 | 0.000 | 0.000 | 1.000 | 0.000 | 1.000 | 0.000 | 1.000 | 0.000 |
| Mole Flow lbmol/hr | 211.238 | 237.976 | 184.592 | 53.384 | 53.378 | 0.005 | 124.957 | 59.635 | 59.629 |
| Mass Flow lb/hr | 6075.000 | 6561.454 | 5597.996 | 963.458 | 963.361 | 0.097 | 4522.037 | 1075.959 | 1075.852 |
| Volume Flow cuft/hr | 87.176 | 95.540 | 84.790 | 21709.893 | 16.922 | 2.277 | 65.174 | 35456.719 | 18.642 |
| Enthalpy MMBtu/hr | −30.680 | −33.928 | −26.853 | −5.489 | −6.454 | −0.001 | −19.747 | −6.140 | −7.234 |
| Mass Flow lb/hr | | | | | | | | | |
| GLYCEROL | 2820.0000 | 2825.9110 | 2824.0910 | 1.8200 | 1.8200 | 0.0000 | 2822.2360 | 1.8550 | 1.8550 |
| WATER | 3253.0000 | 3733.5320 | 2772.5170 | 961.0150 | 960.9200 | 0.0960 | 1698.6920 | 1073.8250 | 1073.7180 |
| NACL | 1.0000 | 1.0000 | 1.0000 | 0.0000 | 0.0000 | 0.0000 | 1.0000 | 0.0000 | 0.0000 |
| METHANOL | 1.0000 | 1.0100 | 0.3880 | 0.6220 | 0.6210 | 0.0010 | 0.1090 | 0.2790 | 0.2790 |
| NITROGEN | 0.0000 | 0.0000 | 0.0000 | 0.0000 | 0.0000 | 0.0000 | 0.0000 | 0.0000 | 0.0000 |
| Mass Frac | | | | | | | | | |
| GLYCEROL | 0.4640 | 0.4310 | 0.5040 | 0.0020 | 0.0020 | 0.0000 | 0.6240 | 0.0020 | 0.0020 |
| WATER | 0.5350 | 0.5690 | 0.4950 | 0.9970 | 0.9970 | 0.9920 | 0.3760 | 0.9980 | 0.9980 |
| NACL | 0.0000 | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0010 | 0.0010 | 0.0070 | 0.0000 | 0.0000 | 0.0000 |
| NITROGEN | 0.0000 | 0.0000 | 0.0000 | 0.0000 | 0.0000 | 0.0010 | 0.0000 | 0.0000 | 0.0000 |
| Mole Flow lbmol/hr | | | | | | | | | |
| GLYCEROL | 30.6210 | 30.6850 | 30.6650 | 0.0200 | 0.0200 | 0.0000 | 30.6450 | 0.0200 | 0.0200 |
| WATER | 180.5690 | 207.2430 | 153.8980 | 53.3440 | 53.3390 | 0.0050 | 94.2920 | 59.6060 | 59.6000 |
| NACL | 0.0170 | 0.0170 | 0.0170 | 0.0000 | 0.0000 | 0.0000 | 0.0170 | 0.0000 | 0.0000 |
| METHANOL | 0.0310 | 0.0320 | 0.0120 | 0.0190 | 0.0190 | 0.0000 | 0.0030 | 0.0090 | 0.0090 |
| NITROGEN | 0.0000 | 0.0000 | 0.0000 | 0.0000 | 0.0000 | 0.0000 | 0.0000 | 0.0000 | 0.0000 |
| Mole Frac | | | | | | | | | |
| GLYCEROL | 0.1450 | 0.1290 | 0.1660 | 0.0000 | 0.0000 | 0.0000 | 0.2450 | 0.0000 | 0.0000 |
| WATER | 0.8550 | 0.8710 | 0.8340 | 0.9990 | 0.9990 | 0.9950 | 0.7550 | 1.0000 | 1.0000 |
| NACL | 0.0000 | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0040 | 0.0000 | 0.0000 | 0.0000 |
| NITROGEN | 0.0000 | 0.0000 | 0.0000 | 0.0000 | 0.0000 | 0.0010 | 0.0000 | 0.0000 | 0.0000 |

| | ST11 | ST12 | ST13 | ST14 | ST15 | ST17 | ST18 | ST19 |
|---|---|---|---|---|---|---|---|---|
| Temperature F. | 201.40 | 188.90 | 188.90 | 166.60 | 166.60 | 166.50 | 188.90 | 188.90 |
| Pressure psi | 11.00 | 6.00 | 6.00 | 5.00 | 5.00 | 5.00 | 6.00 | 30.00 |
| Vapor Frac | 1.000 | 0.000 | 1.000 | 0.000 | 1.000 | 0.030 | 1.000 | 0.000 |
| Mole Flow lbmol/hr | 0.006 | 58.127 | 66.830 | 66.834 | 0.007 | 179.842 | 66.841 | 58.127 |
| Mass Flow lb/hr | 0.108 | 3315.680 | 1206.357 | 1206.441 | 0.121 | 3245.653 | 1206.561 | 3315.680 |
| Volume Flow cuft/hr | 3.820 | 44.289 | 77217.795 | 20.456 | 8.952 | 7203.743 | 77231.257 | 44.289 |
| Enthalpy MMBtu/hr | −0.001 | −11.759 | −6.893 | −8.156 | −0.001 | −21.843 | −6.894 | −11.759 |
| Mass Flow lb/hr | | | | | | | | |
| GLYCEROL | 0.0000 | 2819.3060 | 2.9300 | 2.9300 | 0.0000 | 6.6050 | 2.9300 | 2819.3060 |
| WATER | 0.1070 | 495.3610 | 1203.3310 | 1203.4130 | 0.1200 | 3238.0500 | 1203.5340 | 495.3610 |
| NACL | 0.0000 | 1.0000 | 0.0000 | 0.0000 | 0.0000 | 0.0000 | 0.0000 | 1.0000 |
| METHANOL | 0.0000 | 0.0120 | 0.0960 | 0.0970 | 0.0000 | 0.9980 | 0.0970 | 0.0120 |
| NITROGEN | 0.0000 | 0.0000 | 0.0000 | 0.0000 | 0.0000 | 0.0000 | 0.0000 | 0.0000 |
| Mass Frac | | | | | | | | |
| GLYCEROL | 0.0000 | 0.8500 | 0.0020 | 0.0020 | 0.0000 | 0.0020 | 0.0020 | 0.8500 |
| WATER | 0.9970 | 0.1490 | 0.9970 | 0.9970 | 0.9980 | 0.9980 | 0.9970 | 0.1490 |
| NACL | 0.0000 | 0.0000 | 0.0000 | 0.0000 | 0.0000 | 0.0000 | 0.0000 | 0.0000 |
| METHANOL | 0.0030 | 0.0000 | 0.0000 | 0.0000 | 0.0010 | 0.0000 | 0.0000 | 0.0000 |
| NITROGEN | 0.0000 | 0.0000 | 0.0000 | 0.0000 | 0.0010 | 0.0000 | 0.0000 | 0.0000 |
| Mole Flow lbmol/hr | | | | | | | | |
| GLYCEROL | 0.0000 | 30.6130 | 0.0320 | 0.0320 | 0.0000 | 0.0720 | 0.0320 | 30.6130 |
| WATER | 0.0060 | 27.4970 | 66.7950 | 66.8000 | 0.0070 | 179.7390 | 66.8060 | 27.4970 |
| NACL | 0.0000 | 0.0170 | 0.0000 | 0.0000 | 0.0000 | 0.0000 | 0.0000 | 0.0170 |
| METHANOL | 0.0000 | 0.0000 | 0.0030 | 0.0030 | 0.0000 | 0.0310 | 0.0030 | 0.0000 |
| NITROGEN | 0.0000 | 0.0000 | 0.0000 | 0.0000 | 0.0000 | 0.0000 | 0.0000 | 0.0000 |
| Mole Frac | | | | | | | | |
| GLYCEROL | 0.0000 | 0.5270 | 0.0000 | 0.0000 | 0.0000 | 0.0000 | 0.0000 | 0.5270 |
| WATER | 0.9980 | 0.4730 | 0.9990 | 0.9990 | 0.9990 | 0.9990 | 0.9990 | 0.4730 |
| NACL | 0.0000 | 0.0000 | 0.0000 | 0.0000 | 0.0000 | 0.0000 | 0.0000 | 0.0000 |
| METHANOL | 0.0020 | 0.0000 | 0.0000 | 0.0000 | 0.0010 | 0.0000 | 0.0000 | 0.0000 |
| NITROGEN | 0.0000 | 0.0000 | 0.0000 | 0.0000 | 0.0010 | 0.0000 | 0.0000 | 0.0000 |

| | ST20 | ST21 | ST22 | ST23 | ST24 | ST25 | ST26 | ST27 |
|---|---|---|---|---|---|---|---|---|
| Temperature F. | 275.00 | 170.50 | 100.00 | 100.00 | 225.00 | 250.00 | 100.00 | 100.10 |
| Pressure psi | 25.00 | 16.00 | 15.00 | 15.00 | 27.50 | 26.50 | 15.00 | 25.00 |
| Vapor Frac | 0.080 | 1.000 | 1.000 | 1.000 | 1.000 | 1.000 | 0.000 | 0.000 |
| Mole Flow lbmol/hr | 58.127 | 499.871 | 473.133 | 473.115 | 473.115 | 473.115 | 26.738 | 26.738 |
| Mass Flow lb/hr | 3315.680 | 13486.751 | 13000.298 | 13000.000 | 13000.000 | 13000.000 | 486.454 | 486.454 |
| Volume Flow cuft/hr | 1490.286 | 211126.207 | 189314.941 | 189299.705 | 126374.781 | 135953.812 | 7.918 | 7.919 |
| Enthalpy MMBtu/hr | −11.515 | −5.101 | −2.566 | −2.564 | −2.148 | −2.065 | −3.305 | −3.305 |
| Mass Flow lb/hr | | | | | | | | |
| GLYCEROL | 2819.3060 | 5.9380 | 0.0270 | 0.0270 | 0.0270 | 0.0270 | 5.9110 | 5.9110 |
| WATER | 495.3610 | 937.9060 | 457.3740 | 457.0120 | 457.0120 | 457.0120 | 480.5320 | 480.5320 |
| NACL | 1.0000 | 0.0000 | 0.0000 | 0.0000 | 0.0000 | 0.0000 | 0.0000 | 0.0000 |
| METHANOL | 0.0120 | 0.2040 | 0.1940 | 0.1940 | 0.1940 | 0.1940 | 0.0100 | 0.0100 |
| NITROGEN | 0.0000 | 12542.7020 | 12542.7020 | 12542.7670 | 12542.7670 | 12542.7670 | 0.0000 | 0.0000 |
| Mass Frac | | | | | | | | |
| GLYCEROL | 0.8500 | 0.0000 | 0.0000 | 0.0000 | 0.0000 | 0.0000 | 0.0120 | 0.0120 |
| WATER | 0.1490 | 0.0700 | 0.0350 | 0.0350 | 0.0350 | 0.0350 | 0.9880 | 0.9880 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.9300 | 0.9650 | 0.9650 | 0.9650 | 0.9650 | 0.0000 | 0.0000 |
| Mole Flow lbmol/hr | | | | | | | | |
| GLYCEROL | 30.6130 | 0.0640 | 0.0000 | 0.0000 | 0.0000 | 0.0000 | 0.0640 | 0.0640 |
| WATER | 27.4970 | 52.0620 | 25.3880 | 25.3680 | 25.3680 | 25.3680 | 26.6740 | 26.6740 |
| NACL | 0.0170 | 0.0000 | 0.0000 | 0.0000 | 0.0000 | 0.0000 | 0.0000 | 0.0000 |
| METHANOL | 0.0 000 | 0.0060 | 0.0060 | 0.0060 | 0.0060 | 0.0060 | 0.0000 | 0.0000 |
| NITROGEN | 0.0000 | 447.7380 | 447.7380 | 447.7400 | 447.7400 | 447.7400 | 0.0000 | 0.0000 |
| Mole Frac | | | | | | | | |
| GLYCEROL | 0.5270 | 0.0000 | 0.0000 | 0.0000 | 0.0000 | 0.0000 | 0.0020 | 0.0020 |
| WATER | 0.4730 | 0.1040 | 0.0540 | 0.0540 | 0.0540 | 0.0540 | 0.9980 | 0.9980 |
| NACL | 0.0000 | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 | 0.0000 |
| NITROGEN | 0.0000 | 0.8960 | 0.9460 | 0.9460 | 0.9460 | 0.9460 | 0.0000 | 0.0000 |

| | ST28 | ST29 | ST30 | ST32 | ST33 | ST35 | ST40 |
|---|---|---|---|---|---|---|---|
| Temperature F. | 210.40 | 100.00 | 235.40 | 235.40 | 195.70 | 100.00 | 100.00 |
| Pressure psi | 25.00 | 25.00 | 16.50 | 20.00 | 20.00 | 18.00 | 15.00 |
| Vapor Frac | 0.000 | 1.000 | 0.000 | 0.000 | 0.000 | 0.000 | 1.000 |
| Mole Flow lbmol/hr | 26.738 | 0.357 | 31.371 | 31.371 | 31.371 | 31.371 | 0.375 |
| Mass Flow lb/hr | 486.454 | 10.000 | 2828.929 | 2828.929 | 2828.929 | 2828.929 | 10.297 |
| Volume Flow cuft/hr | 8.454 | 85.715 | 37.718 | 37.718 | 37.144 | 36.126 | 149.946 |
| Enthalpy MMBtu/hr | −3.247 | 0.000 | −8.479 | −8.479 | −8.537 | −8.671 | −0.002 |
| Mass Flow lb/hr | | | | | | | |
| GLYCEROL | 5.9110 | 0.0000 | 2813.3950 | 2813.3950 | 2813.3950 | 2813.3950 | 0.0000 |
| WATER | 480.5320 | 0.0000 | 14.4670 | 14.4670 | 14.4670 | 14.4670 | 0.3620 |
| NACL | 0.0000 | 0.0000 | 1.0000 | 1.0000 | 1.0000 | 1.0000 | 0.0000 |
| METHANOL | 0.0100 | 0.0000 | 0.0020 | 0.0020 | 0.0020 | 0.0020 | 0.0000 |
| NITROGEN | 0.0000 | 10.0000 | 0.0650 | 0.0650 | 0.0650 | 0.0650 | 9.9350 |
| Mass Frac | | | | | | | |
| GLYCEROL | 0.0120 | 0.0000 | 0.9950 | 0.9950 | 0.9950 | 0.9950 | 0.0000 |
| WATER | 0.9880 | 0.0000 | 0.0050 | 0.0050 | 0.0050 | 0.0050 | 0.0350 |
| NACL | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 |
| NITROGEN | 0.0000 | 1.0000 | 0.0000 | 0.0000 | 0.0000 | 0.0000 | 0.9650 |
| Mole Flow lbmol/hr | | | | | | | |
| GLYCEROL | 0.0640 | 0.0000 | 30.5490 | 30.5490 | 30.5490 | 30.5490 | 0.0000 |
| WATER | 26.6740 | 0.0000 | 0.8030 | 0.8030 | 0.8030 | 0.8030 | 0.0200 |
| NACL | 0.0000 | 0.0000 | 0.0170 | 0.0170 | 0.0170 | 0.0170 | 0.0000 |
| METHANOL | 0.0000 | 0.0000 | 0.0000 | 0.0000 | 0.0000 | 0.0000 | 0.0000 |
| NITROGEN | 0.0000 | 0.3570 | 0.0020 | 0.0020 | 0.0020 | 0.0020 | 0.3550 |
| Mole Frac | | | | | | | |
| GLYCEROL | 0.0020 | 0.0000 | 0.9740 | 0.9740 | 0.9740 | 0.9740 | 0.0000 |
| WATER | 0.9980 | 0.0000 | 0.0260 | 0.0260 | 0.0260 | 0.0260 | 0.0540 |
| NACL | 0.0000 | 0.0000 | 0.0010 | 0.0010 | 0.0010 | 0.0010 | 0.0000 |
| METHANOL | 0.0000 | 0.0000 | 0.0000 | 0.0000 | 0.0000 | 0.0000 | 0.0000 |
| NITROGEN | 0.0000 | 1.0000 | 0.0000 | 0.0000 | 0.0000 | 0.0000 | 0.9460 |

TABLE 5

| | ST01 | ST03 | ST04 | ST05 | ST06 | ST08 | ST09 |
|---|---|---|---|---|---|---|---|
| Temperature F. | 100.00 | 229.70 | 229.70 | 222.50 | 222.50 | 210.50 | 210.50 |
| Pressure psi | 25.00 | 18.00 | 18.00 | 17.00 | 17.00 | 12.00 | 12.00 |
| Vapor Frac | 0.000 | 0.000 | 1.000 | 0.000 | 1.000 | 0.000 | 1.000 |
| Mole Flow lbmol/hr | 211.238 | 166.308 | 44.930 | 44.925 | 0.004 | 115.563 | 50.745 |
| Mass Flow lb/hr | 6075.000 | 5264.035 | 810.965 | 810.884 | 0.081 | 4348.389 | 915.646 |
| Volume Flow cuft/hr | 87.176 | 78.928 | 18281.385 | 14.244 | 1.916 | 62.209 | 30194.085 |
| Enthalpy MMBtu/hr | −30.680 | −24.643 | −4.619 | −5.432 | 0.000 | −18.606 | −5.225 |
| Mass Flow lb/hr | | | | | | | |
| GLYCEROL | 2820.0000 | 2818.4040 | 1.5960 | 1.5960 | 0.0000 | 2816.7540 | 1.6510 |
| WATER | 3253.0000 | 2444.2260 | 808.7740 | 808.6940 | 0.0810 | 1530.5150 | 913.7110 |
| NACL | 1.0000 | 1.0000 | 0.0000 | 0.0000 | 0.0000 | 1.0000 | 0.0000 |
| METHANOL | 1.0000 | 0.4060 | 0.5940 | 0.5940 | 0.0010 | 0.1210 | 0.2850 |
| Mass Frac | | | | | | | |
| GLYCEROL | 0.4640 | 0.5350 | 0.0020 | 0.0020 | 0.0000 | 0.6480 | 0.0020 |
| WATER | 0.5350 | 0.4640 | 0.9970 | 0.9970 | 0.9920 | 0.3520 | 0.9980 |
| NACL | 0.0000 | 0.0000 | 0.0000 | 0.0000 | 0.0000 | 0.0000 | 0.0000 |
| METHANOL | 0.0000 | 0.0000 | 0.0010 | 0.0010 | 0.0080 | 0.0000 | 0.0000 |
| Mole Flow lbmol/hr | | | | | | | |
| GLYCEROL | 30.6210 | 30.6030 | 0.0170 | 0.0170 | 0.0000 | 30.5850 | 0.0180 |
| WATER | 180.5690 | 135.6750 | 44.8940 | 44.8890 | 0.0040 | 84.9560 | 50.7190 |
| NACL | 0.0170 | 0.0170 | 0.0000 | 0.0000 | 0.0000 | 0.0170 | 0.0000 |
| METHANOL | 0.0310 | 0.0130 | 0.0190 | 0.0190 | 0.0000 | 0.0040 | 0.0090 |
| Mole Frac | | | | | | | |
| GLYCEROL | 0.1450 | 0.1840 | 0.0000 | 0.0000 | 0.0000 | 0.2650 | 0.0000 |
| WATER | 0.8550 | 0.8160 | 0.9990 | 0.9990 | 0.9960 | 0.7350 | 0.9990 |
| NACL | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0040 | 0.0000 | 0.0000 |

| | ST10 | ST11 | ST12 | ST13 | ST14 | ST15 | ST17 | ST18 |
|---|---|---|---|---|---|---|---|---|
| Temperature F. | 201.40 | 201.40 | 188.80 | 188.80 | 166.60 | 166.60 | 166.50 | 188.80 |
| Pressure psi | 11.00 | 11.00 | 6.00 | 6.00 | 5.00 | 5.00 | 5.00 | 6.00 |
| Vapor Frac | 0.000 | 1.000 | 0.000 | 1.000 | 0.000 | 1.000 | 0.029 | 1.000 |
| Mole Flow lbmol/hr | 50.740 | 0.005 | 58.076 | 57.487 | 57.491 | 0.006 | 153.156 | 57.497 |
| Mass Flow lb/hr | 915.555 | 0.092 | 3310.676 | 1037.713 | 1037.782 | 0.104 | 2764.220 | 1037.886 |
| Volume Flow cuft/hr | 15.864 | 3.250 | 44.224 | 66418.798 | 17.597 | 7.701 | 6095.274 | 66428.531 |
| Enthalpy MMBtu/hr | −6.156 | −0.001 | −11.745 | −5.929 | −7.015 | −0.001 | −18.603 | −5.930 |
| Mass Flow lb/hr | | | | | | | | |
| GLYCEROL | 1.6510 | 0.0000 | 2814.2380 | 2.5150 | 2.5150 | 0.0000 | 5.7620 | 2.5150 |
| WATER | 913.6200 | 0.0910 | 495.4230 | 1035.0920 | 1035.1600 | 0.1030 | 2757.4740 | 1035.2640 |
| NACL | 0.0000 | 0.0000 | 1.0000 | 0.0000 | 0.0000 | 0.0000 | 0.0000 | 0.0000 |
| METHANOL | 0.2840 | 0.0000 | 0.0160 | 0.1050 | 0.1060 | 0.0000 | 0.9840 | 0.1060 |
| Mass Frac | | | | | | | | |
| GLYCEROL | 0.0020 | 0.0000 | 0.8500 | 0.0020 | 0.0020 | 0.0000 | 0.0020 | 0.0020 |
| WATER | 0.9980 | 0.9960 | 0.1500 | 0.9970 | 0.9970 | 0.9990 | 0.9980 | 0.9970 |
| NACL | 0.0000 | 0.0000 | 0.0000 | 0.0000 | 0.0000 | 0.0000 | 0.0000 | 0.0000 |
| METHANOL | 0.0000 | 0.0040 | 0.0000 | 0.0000 | 0.0000 | 0.0010 | 0.0000 | 0.0000 |
| Mole Flow lbmol/hr | | | | | | | | |
| GLYCEROL | 0.0180 | 0.0000 | 30.5580 | 0.0270 | 0.0270 | 0.0000 | 0.0630 | 0.0270 |
| WATER | 50.7140 | 0.0050 | 27.5000 | 57.4560 | 57.4600 | 0.0060 | 153.0630 | 57.4660 |
| NACL | 0.0000 | 0.0000 | 0.0170 | 0.0000 | 0.0000 | 0.0000 | 0.0000 | 0.0000 |
| METHANOL | 0.0090 | 0.0000 | 0.0000 | 0.0030 | 0.0030 | 0.0000 | 0.0310 | 0.0030 |
| Mole Frac | | | | | | | | |
| GLYCEROL | 0.0000 | 0.0000 | 0.5260 | 0.0000 | 0.0000 | 0.0000 | 0.0000 | 0.0000 |
| WATER | 0.9990 | 0.9980 | 0.4740 | 0.9990 | 0.9990 | 0.9990 | 0.9990 | 0.9990 |
| NACL | 0.0000 | 0.0000 | 0.0000 | 0.0000 | 0.0000 | 0.0000 | 0.0000 | 0.0000 |
| METHANOL | 0.0000 | 0.0020 | 0.0000 | 0.0000 | 0.0000 | 0.0010 | 0.0000 | 0.0000 |

| | ST19 | ST20 | ST30 | ST32 | ST33 | ST34 | ST35 |
|---|---|---|---|---|---|---|---|
| Temperature F. | 188.80 | 150.00 | 333.40 | 333.60 | 200.00 | 149.10 | 130.00 |
| Pressure psi | 30.00 | 25.00 | 3.50 | 20.00 | 19.00 | 2.50 | 2.00 |
| Vapor Frac | 0.000 | 0.000 | 0.000 | 0.000 | 0.000 | 1.000 | 0.000 |
| Mole Flow lbmol/hr | 58.076 | 58.076 | 31.291 | 31.291 | 31.291 | 26.785 | 26.785 |
| Mass Flow lb/hr | 3310.676 | 3310.676 | 2827.669 | 2827.669 | 2827.669 | 483.007 | 483.007 |
| Volume Flow cuft/hr | 44.224 | 43.514 | 39.249 | 39.252 | 37.187 | 69856.544 | 8.018 |
| Enthalpy MMBtu/hr | −11.744 | −11.819 | −8.321 | −8.321 | −8.522 | −2.770 | −3.287 |
| Mass Flow lb/hr | | | | | | | |
| GLYCEROL | 2814.2380 | 2814.2380 | 2813.6690 | 2813.6690 | 2813.6690 | 0.5690 | 0.5690 |
| WATER | 495.4230 | 495.4230 | 13.0000 | 13.0000 | 13.0000 | 482.4230 | 482.4230 |
| NACL | 1.0000 | 1.0000 | 1.0000 | 1.0000 | 1.0000 | 0.0000 | 0.0000 |
| METHANOL | 0.0160 | 0.0160 | 0.0000 | 0.0000 | 0.0000 | 0.0160 | 0.0160 |
| Mass Frac | | | | | | | |
| GLYCEROL | 0.8500 | 0.8500 | 0.9950 | 0.9950 | 0.9950 | 0.0010 | 0.0010 |
| WATER | 0.1500 | 0.1500 | 0.0050 | 0.0050 | 0.0050 | 0.9990 | 0.9990 |
| NACL | 0.0000 | 0.0000 | 0.0000 | 0.0000 | 0.0000 | 0.0000 | 0.0000 |
| METHANOL | 0.0000 | 0.0000 | 0.0000 | 0.0000 | 0.0000 | 0.0000 | 0.0000 |
| Mole Flow lbmol/hr | | | | | | | |
| GLYCEROL | 30.5580 | 30.5580 | 30.5520 | 30.5520 | 30.5520 | 0.0060 | 0.0060 |
| WATER | 27.5000 | 27.5000 | 0.7220 | 0.7220 | 0.7220 | 26.7790 | 26.7790 |
| NACL | 0.0170 | 0.0170 | 0.0170 | 0.0170 | 0.0170 | 0.0000 | 0.0000 |
| METHANOL | 0.0000 | 0.0000 | 0.0000 | 0.0000 | 0.0000 | 0.0000 | 0.0000 |
| Mole Frac | | | | | | | |
| GLYCEROL | 0.5260 | 0.5260 | 0.9760 | 0.9760 | 0.9760 | 0.0000 | 0.0000 |
| WATER | 0.4740 | 0.4740 | 0.0230 | 0.0230 | 0.0230 | 1.0000 | 1.0000 |
| NACL | 0.0000 | 0.0000 | 0.0010 | 0.0010 | 0.0010 | 0.0000 | 0.0000 |
| METHANOL | 0.0000 | 0.0000 | 0.0000 | 0.0000 | 0.0000 | 0.0000 | 0.0000 |

Although the preferred embodiment of the present invention has been described herein with reference to the accompanying drawings and examples, it is to be understood that the invention is not limited to that precise embodiment or examples, and that various other changes and modifications may be affected therein by one skilled in the art without departing from the scope or spirit of the invention.

What is claimed is:

1. A process for purifying crude glycerin comprising the steps of:
a) providing crude glycerin, said crude glycerin comprising glycerol, water, and at least one of methanol, free fatty acids, FAME, and salts;
b) fractionating the crude glycerin by ion exclusion chromatographic separation thereby forming at least a first fraction comprising glycerol, water, and salt, and a second fraction comprising water and at least one of methanol, free fatty acids, FAME, and further salts, wherein said fractionating comprises contacting the crude glycerin with a first ion exchange resin capable of ion exclusion chromatographic separation thereby separating the glycerol from the at least one of methanol, free fatty acids, FAME, and further salts;
c) a first dewatering of the first fraction thereby producing an industrial grade glycerin solution product, said industrial grade glycerin solution product comprising glycerol and water where the glycerol weight percent is 60 to 95 wt %; and
d) a second dewatering of the industrial grade glycerin solution product thereby producing a purified grade glycerin solution product comprising glycerol and water where the glycerol weight percent is between 95 and 99 wt %.

2. The process according to claim 1, wherein the second dewatering step comprises adding the industrial grade glycerin solution to a glycerin water stripper apparatus having a bottom, a middle, and a top area, in which recirculating nitrogen gas and/or air is introduced into the bottom and wherein water of the industrial grade glycerin solution is removed from the middle and/or top of the apparatus while the purified grade glycerin solution product is collected in the bottom of the glycerin water stripper apparatus.

3. The process according to claim 2, wherein the ion exclusion chromatographic separation comprises the use of a single column fixed bed process.

4. The process according to claim 2, wherein the ion exclusion chromatographic separation comprises the use of a simulated moving bed process.

5. The process according to claim 2, wherein the ion exclusion chromatographic separation comprises the use of a moving bed process.

6. The process according to claim 2, further comprising, after the ion exclusion chromatographic separation is performed, contacting the first fraction with at least one further ion exchange resin whereby salt of the first fraction is removed.

7. The process according to claim 2, further comprising contacting the purified grade glycerin product solution with activated carbon thereby reducing odor and/or color.

8. The process according to claim 2, wherein said fractionation is monitored and/or controlled by means of refractive index and conductivity testing.

9. The process according to claim 2, wherein said fractionation is monitored and/or controlled by on-line gas chromatography and on-line ion chromatography.

10. The process according to claim 2, wherein said first dewatering step comprises evaporating the first fraction by means of multi-effect flash vacuum evaporation.

11. The process according to claim 2, wherein said first dewatering step comprises performing a multiple stage flash evaporation with vacuum or at atmospheric pressure.

12. The process in claim 1, wherein second dewatering step comprises adding the industrial grade glycerin solution to a multi-effect vacuum evaporation apparatus whereby water of the industrial grade glycerin solution is evaporated.

13. The process in claim 1, wherein the second dewatering step comprises adding the industrial grade glycerin solution to a thermal recompression apparatus whereby water of the industrial grade glycerin solution is evaporated.

14. The process in claim 1, wherein the second dewatering step comprises adding the industrial grade glycerin solution to a reboiled distillation apparatus whereby water of the industrial grade glycerin solution is evaporated.
